SEQUENCE LISTING (8 sequences)

SEQ ID NO: 1 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 2 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 3 - protein, 188 aa, Homo sapiens [sequence omitted]

SEQ ID NO: 4 - DNA, 3159 bp, Homo sapiens
cgctccaccagatccgcgggagccccactgctctccgggtccttggcttgtggctgtgggtcccatcgggcccgccctcgcacgtcactccgggacccccgcggcctccgcaggttctgcgctccaggccggagtcagagactccaggatcggttctttcatcttcgccgcccctgcgcgtccagctcttctaagacgagatgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaatgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactgaggggccaggcaggggtgggagagccagctctctctacccggcccagagaccccttcctttcctctgcagcactttaaccctctcttccctattatttccgggtggaaaagaatccctgcagcctggtagaattgggaagctgggggaagggtggtctgagcaccccctcattcccctcgtgtgactctcttggattatttatgtgttgtggtttggccgtggccatcagggtgggccactctcccctccctcttccttcccccatcccctttcctccccaccttccccagactcagctccagaataccttcttcgctgctagagaagggggattggagggaagacaggtctagactttctcagtgggacaaaccagagcagagagcaggacaggagacaagaaatccagtttcccaccaccttggactcctcccacaatctgggactttcactgaattcttgccacgcagactctgggcaaaggggtttttttttttttttttttttttttttttgagacagtctcgctctgtcgcccaggctcgagtgcagtggcgtgatcttgcttcactgcaagctgtctcccaggttcacgccattctcctgcctcagcctccggagtagctgggactacaggcgcatgccaccacacctggctaatttattttgtatttttagtatatacgcggtttcaccatgttagccagaatggtctcgatctcctgacctcgtgatctgcctgcctcagcctcccaaagtgctgggattacaggcgtgagccaccgcgcctggcgaagggagttctctcttgacccctgcagggaaaaggactcacctccctcactgcaggctcagccttccagggcaagagggaacaggaaagtatgtgcccatgtgtggcaagatggaaggacggcaggctcccgcctctaggcttggggctctaccccgatggtttcccaaggctgccaagaaggagccctaactttcttcctctcccttcctggaagggtgctgcatccacaggcttttgaccaactaaggcaaagaggggatttgaaaggctgcctggaaacactgggctgggaggagcctttggatatttttatatacgtttgaaaaggggattgagagaagaaaccaaaggtcggttgtactaaatgtatatatatagatacttctataaagtcactgctgaagacaagcatcctattgtggaggtacttgaggatgggctgagacagggaccataactcttcacccctcttcctccctctgtcctgcctcagctcaaggcctcagaatcttctggatgccattgctcatgcccctactcacatttctactcgttgctttattaatagtaaatgctcaataaattgtagctgccagtgccgggcattgctcttggcatttgcaaaaaaaaaaa

SEQ ID NO: 5 - DNA, 1530 bp, Homo sapiens
atgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaatgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactga

SEQ ID NO: 6 - protein, 509 aa, Homo sapiens
MPSGFQQIGSEDGEPPQQRVTGTLVLAVFSAVLGSLQFGYNIGVINAPQKVIEQSYNETWLGRQGPEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 7 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 8 - protein, 1210 aa, Homo sapiens [sequence omitted]

(19) United States
(12) Patent Application Publication
(10) Pub. No.: US 2017/0035917 A1
(43) Pub. Date: Feb. 9, 2017

(54) METHODS OF TREATMENT OF CANCER

(71) Applicant: MILLENNIUM PHARMACEUTICALS, INC., Cambridge, MA (US)

(72) Inventors: Daniel P. BRADLEY, Somerville, MA (US); Robbie J. ROBERSTON, Somerville, MA (US)

(73) Assignee: Millennium Pharmaceuticals, Inc., Cambridge, MA (US)

(21) Appl. No.: 14/896,013

(22) PCT Filed: Jun. 10, 2014
(86) PCT No.: PCT/US14/41643

§ 371 Date: Dec. 4, 2015

Related U.S. Application Data
(60) Provisional application No. 61/833,186, filed on Jun. 10, 2013.

(51) Int. Cl. A61K 51/10; A61K 31/69; A61K 49/06; A61K 49/04

(52) U.S. Cl. CPC A61K 51/1096; A61K 49/04; A61K 31/69; A61K 49/06

(57) ABSTRACT

The present invention provides a method of treating cancer with a proteasome inhibitor. The invention provides a method for treating a patient with a proteasome inhibitor based on measurements of tumor features using biomedical imaging techniques. The invention also provides a method of treating a patient with cancer based on levels of GLUT4, as measured by a biomedical imaging technique. The invention also provides a method of treating a cancer patient with a proteasome inhibitor based on the effect of the treatment on tumor features measured by a biomedical imaging technique.

### RELATED APPLICATIONS

This application claims priority to U.S. Provisional Application No. 61/833,186 filed on Jun. 10, 2013. The entire contents of the foregoing application are incorporated herein by reference.

### SEQUENCE LISTING

The contents of the Sequence Listing are submitted herewith electronically in duplicate. Each duplicate has a copy of the Sequence Listing file, created on Jun. 4, 2014 and named “sequencelisting.txt,” and “sequencelisting.pdf” and has a size of 35.1 kb (35,975 bytes). The entire contents of the Sequence Listing in the electronic sequencelisting.txt file are incorporated herein by this reference.

### FIELD OF THE INVENTION

The present invention provides a method of treating cancer with a proteasome inhibitor.

### BACKGROUND OF THE INVENTION

Cancer is a cellular disorder characterized by uncontrolled or disregulated cell proliferation, decreased cellular differentiation, inappropriate ability to invade surrounding tissue, and/or ability to establish new growth at ectopic sites. Depending on the specific cancer involved, the treatment for cancer may involve surgery, radiotherapy, and chemotherapy. There remains a continuing need for new and improved treatments for patients with cancer.

Proteasome inhibition represents an important new strategy in cancer treatment. King et al., Science 274:1652-1659 (1996), describes an essential role for the ubiquitin-proteasome pathway in regulating cell cycle, neoplastic growth and metastasis. The authors teach that a number of key regulatory proteins, including cyclins, and the cyclin-dependent kinases p21 and p27^{KIP1}, are temporally degraded during the cell cycle by the ubiquitin-proteasome pathway. The ordered degradation of these proteins is required for the cell to progress through the cell cycle and to undergo mitosis.

The proteasome inhibitor VELCADE® (bortezomib; N-2-pyrazinecarbonyl-L-phenylalanine-L-leucineboronic acid) is the first proteasome inhibitor to achieve regulatory approval. Mitsiades et al., Current Drug Targets, 7:1341 (2006), reviews the clinical studies leading to the approval of bortezomib for the treatment of multiple myeloma patients who have received at least one prior therapy. Fisher et al., J. Clin. Oncol., 30:4867, describes an international multi-center Phase II study confirming the activity of bortezomib in patients with relapsed or refractory mantle cell lymphoma. Ishii et al., Anti-Cancer Agents in Medicinal Chemistry, 7:359 (2007), and Roccaro et al., Curr. Pharm. Biotech., 7:1341 (2006), discuss a number of molecular mechanisms that may contribute to the antitumor activities of bortezomib. The proteasome inhibitor MLN9708 [2,2′-{2-[(1R)-1-({[(2,5-dichlorobenzoy)amino]acetyl}amino)-3-methylbutyl]-5-oxo-1,3,2-dioxaborolane-4,4-diyl}diacetic acid] is currently undergoing clinical evaluation for hematological and solid cancers. MLN9708 is a citrate ester which rapidly hydrolyzes to the active form [(1R)-1-({[(2,5-dichlorobenzoyl)amino]acetyl}amino)-3-methylbutyl]boronic acid (MLN2238) on exposure to aqueous solution or plasma. MLN9708 has demonstrated anti-tumor activity in a range of hematological and solid tumor xenograft models (Kupperman et al. (2010) Cancer Res. 70:1970-1980). There remains a further need to identify cancer patients most likely to benefit from treatment with a proteasome inhibitor.

### SUMMARY

Typical methods to determine extent of cancer or outcome of therapy can employ invasive methods, such as biopsy to collect tumor tissue for genotype or phenotype, e.g., histological analysis. The invention provides noninvasive methods for determining, assessing, advising or providing an appropriate therapy regimen for treating a tumor or managing disease in a patient. Monitoring a treatment using the kits and methods disclosed herein can identify the potential for unfavorable outcome and allow their prevention, and thus a savings in morbidity, mortality and treatment costs through adjustment in the therapeutic regimen, cessation of therapy or use of combination therapy.

The invention relates to treatment of cancer with MLN9708 based on noninvasive biomedical imaging results. In one aspect, the invention relates to the understanding of physical and physiological quantities measured for cancers which are responsive to MLN9708. In one embodiment, the invention relates to a quantity of metabolic activity of tumors which are responsive to MLN9708. In another aspect, the invention relates to a decrease in the metabolic activity of tumors which are responsive to MLN9708. In another aspect, the invention relates to an increase in diffusivity of tumors which are responsive to MLN9708. Accordingly, the invention features treating cancer patients with MLN9708 if a non-invasive measurement of the patient tumor demonstrates responsiveness to MLN9708.

The entire contents of all publications, patent applications, patents and other references mentioned herein are incorporated by reference.

Other features and advantages of the invention will be apparent from the following detailed description, drawings and from the claims.

### DESCRIPTION OF FIGURES

FIG. 1 shows FDG-PET images of representative primary human lung adenocarcinoma xenograft tumors before and 48 h after administration of MLN2238. PHTX132 tumor was responsive, PHTX192 was nonresponsive.

FIGS. 2A-2D show quantification, measured by FDG-PET, of mean values (+/−) SEM and normalized to baseline) SUVaverage (FIGS. 2A and 2C) and SUVmaximum (FIGS. 2B and 2D) for the groups of animals bearing primary human lung adenocarcinoma xenograft tumors, the responsive PHTX132 and the nonresponsive PHTX192.

FIG. 3 shows a time course of effect of MLN9708 treatment on primary human lung adenocarcinoma xenograft tumors PHTX132 (11 mg/kg) measured by FDG-PET. The mean SUVave was significantly decreased by 48 h after treatment.

FIG. 4 shows a time course of effect of MLN9708 treatment on primary human lung adenocarcinoma xenograft tumors PHTX132 and PHTX24C, represented as mean SUVave measured by FDG-PET.

FIG. 5 shows a time course of effect of MLN9708 treatment on primary human lung adenocarcinoma xenograft tumors PHTX192, represented as mean SUVave measured by FDG-PET.

FIG. 6 shows a time course of effect of MLN9708 treatment on tumors grown, from the isogenic cell line SW48 (FIG. 6A) or SW48 with a G13D mutation in KRAS (FIG. 6B), represented as mean SUVave measured by FDG-PET.

FIG. 7 shows a time course of effect of MLN9708 treatment on 3-dimensional in vitro cultures (OTOC) grown from the colon cancer HT29, colon cancer HCT-116 and lung cancer H460 cell lines. The percent change from baseline FDG uptake was measured by Cerenkov luminescence imaging.

FIG. 8 shows a time course of the effect of MLN9708 treatment on 3-dimensional in vitro cultures (OTOC) grown from the isogenic cell line SW48 (FIG. 6A) or SW48 with a G13D mutation in KRAS. The percent change from baseline FDG uptake was measured by Cerenkov luminescence imaging.

FIG. 9 shows a time course of effect of MLN2238 treatment on primary human lung adenocarcinoma xenograft tumors PHTX192, represented as mean SUVave measured by FDG-PET.

FIG. 10 shows a time course of effect of MLN2238 treatment on primary human lung adenocarcinoma xenograft tumors PHTX132, represented as mean SUVave measured by FDG-PET.

FIG. 11 shows a time course of effect of MLN2238 treatment on WSU-DLCL2 xenograft tumors, represented as mean SUVave measured by FDG-PET.

FIG. 12 shows a time course of effect of MLN2238 treatment on PHTX-9C xenograft primary human colon tumors, represented as mean SUVave measured by FDG-PET.

### DESCRIPTION OF THE INVENTION

One of the continued problems with therapy in cancer patients is individual differences in response to therapies. While advances in development of successful cancer therapies progress, only a subset of patients respond to any particular therapy. With the narrow therapeutic index and the toxic potential of many available cancer therapies, such differential responses potentially contribute to patients undergoing unnecessary, ineffective and even potentially harmful therapy regimens. If a designed therapy could be optimized to treat individual patients, such situations could be reduced or even eliminated. Additionally, determining whether a patient is responsive to treatment early in the course of therapy can provide early opportunity to adjust the treatment plan for successful patient therapy overall. Accordingly, there is a need to identify particular cancer patients who are expected to have a favorable outcome when administered particular cancer therapies as well as particular cancer patients who may have a favorable outcome using more aggressive and/or alternative cancer therapies, e.g., alternative to previous or current cancer therapies administered to the patient. It would therefore be beneficial to provide for the diagnosis, prognosis, and monitoring of cancer patients, including, hematological cancer patients, e.g., patients with liquid tumors (such as lymphoma, leukemia and myeloma) and non-hematological cancer patients, e.g., patients with solid tumors (e.g., non-small cell lung cancer, colon cancer, pancreatic cancer, breast cancer, ovarian cancer, melanoma, head and neck carcinoma, prostate cancer or renal cell carcinoma) who would benefit from particular cancer inhibition therapies as well as those who would benefit from a more aggressive and/or alternative cancer inhibition therapy, e.g., alternative to a cancer therapy or therapies the patient is receiving or has received, thus resulting in appropriate preventative measures.

The present invention provides a method for treating cancer with a proteasome inhibitor such as MLN9708, wherein the cancer is characterized by measurement of a physical and/or physiological quantity by one or more than one biomedical imaging technique. In some embodiments, the present invention provides a method of treating cancer, comprising administering a therapeutically effective amount of a proteasome inhibitor, or a pharmaceutically acceptable salt or pharmaceutical composition thereof, to a cancer patient whose primary or metastatic tumor image is characterized as having a low quantity of the feature being measured. In some embodiments, the present invention provides a method of treating cancer, comprising measuring a quantity of a feature of the patient's tumor by a biomedical imaging technique, administering a therapeutically effective amount of a proteasome inhibitor, or a pharmaceutically acceptable salt or pharmaceutical composition thereof, to the cancer patient, measuring the quantity of the feature of the tumor at least one more time, then continuing treatment or modifying the treatment based on difference between the quantities. In some embodiments, the feature is physical, such as surface characteristic or diffusivity. In other embodiments, the feature is physiological, such as metabolic activity or metabolic capacity.

In one aspect, the present invention provides a method for treating cancer comprising measuring a quantity of a feature of the cancer by a biomedical imaging technique, wherein the cancer comprises a solid tumor, or area detectable and quantifiable with a biomedical imaging technique and administering a therapeutically effective amount of the compound of formula (I):

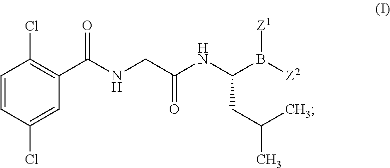

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient if the measurement indicates responsiveness to the compound.

In one embodiment, the solid tumor is from lung cancer. In another embodiment, the solid tumor is from colon cancer.

In one embodiment, the cancer comprises a tumor whose genotype comprises wild type KRAS (v-Ki-ras2 Kirsten rat sarcoma viral oncogene homolog). In one embodiment, the tumor has wild type KRAS or KRAS with mutated codon 146.

In one embodiment, the feature is metabolic capacity. In such an embodiment, the quantity can be the amount of glucose transporter 4 (GLUT4) expression. In another embodiment, the feature is metabolic activity and the quantity is the amount of glucose uptake or amount of metabolites. In another embodiment, the feature is the tumor surface and the quantity is the amount of roughness of the tumor surface. In some embodiments, the quantity is low in a patient whose cancer is responsive to the compound (I) or pharmaceutical composition thereof. For example, a responsive cancer can have tumor with low GLUT4 quantity. In another example, a responsive cancer can have a tumor with low metabolic activity. In another example, a responsive cancer can have a low quantity of surface roughness. In an embodiment, the quantity can be determined as low based on prior experience with imaging a cancer of a similar type as found in the patient. In another embodiment, the quantity can be determined as low based on measurement of a noncancerous tissue in the patient. In another embodiment, the quantity can be determined as low based on assay standards or instrument calibration ranges.

In one aspect, the present invention provides a method for treating cancer, comprising measuring a quantity of the metabolic activity of the cancer by a biomedical imaging technique and administering a therapeutically effective amount of the compound of formula (I):

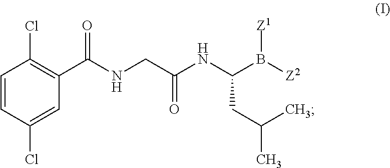

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient if the quantity of metabolic activity is low.

In some embodiments, the method for treating cancer comprises measuring a quantity of a feature of the cancer by a biomedical imaging technique and administering a therapeutically effective amount of the compound of formula (III-A):

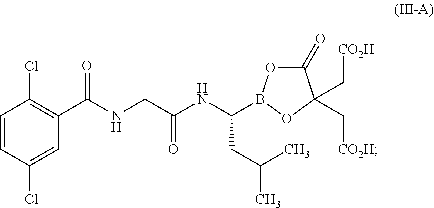 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof;
to the patient if the quantity is low.

In one embodiment, the feature is metabolic capacity. In such an embodiment the quantity can be the amount of glucose transporter 4 (GLUT4) expression. In another embodiment, the feature is metabolic activity. In such an embodiment the quantity can be the uptake or amount of sugar, such as glucose or glycogen, or the amount of metabolite, such as lactate. In another embodiment, the feature is the tumor surface and the quantity can be roughness. In some embodiments, the quantity is low in a patient whose cancer is responsive to the compound (III-A) or pharmaceutical composition thereof. For example, a responsive cancer can have tumor with low GLUT4 quantity. In another example, a responsive cancer can have a tumor with low metabolic activity quantity. In another example, a responsive cancer can have a low quantity of surface roughness. In an embodiment, the quantity can be determined as low based on prior experience with imaging a cancer of a similar type as found in the patient. In another embodiment, the quantity can be determined as low based on measurement of a noncancerous tissue in the patient. In another embodiment, the quantity can be determined as low based on assay standards or instrument calibration ranges.

In some embodiments, the method for treating cancer, comprises measuring a quantity of the metabolic activity of the cancer by a biomedical imaging technique and administering a therapeutically effective amount of the compound of formula (III-A):

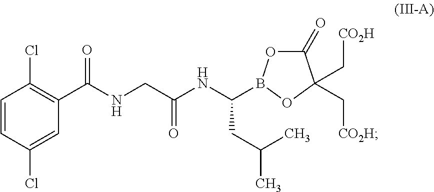 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof;
to the patient if the quantity of metabolic activity is low.

In one embodiment, the cancer is lung cancer. In another embodiment, the cancer is colon cancer.

In one aspect, the present invention provides a method for treating cancer comprising measuring a quantity of a feature of the cancer by a biomedical imaging technique, administering a therapeutically effective amount of the compound of formula (I):

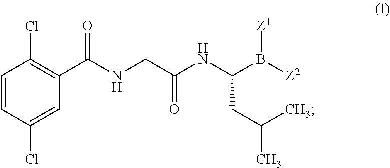

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient, repeating the measurement, and administering one of three types of treatment based on the relationship of the quantity in the second measurement to the quantity in the first measurement, i.e., whether the quantity has decreased, e.g., whether the quantity in the second measurement is lower than in the first measurement.

In one embodiment, the cancer comprises a solid tumor. In one embodiment, the solid tumor is from lung cancer. In another embodiment, the solid tumor is from colon cancer. In another embodiment, the cancer comprises a hematological tumor. In one embodiment, the hematological tumor is lymphoma, such as diffuse large cell lymphoma (DLCL). In another embodiment, the cancer comprises a tumor whose genotype comprises wild type KRAS (v-Ki-ras2 Kirsten rat sarcoma viral oncogene homolog). In one embodiment, the tumor has wild type KRAS or KRAS with mutated codon 146.

In an embodiment, the second measurement of the quantity is early after administering the compound of formula (I), salt, composition or boronic acid anhydride thereof. In the embodiment, the time between administering the compound of formula (I) and the second measurement is short. In some embodiments, the measurement is repeated again. In some embodiments, the additional repeat measurement or measurements are performed before another dose of the compound of formula (I). The additional repeat measurement can confirm the second measurement.

In one embodiment, the feature is metabolic capacity. In such an embodiment the quantity can be the amount of glucose transporter 4 (GLUT4) expression. In another embodiment, the feature is metabolic activity. In such an embodiment, the quantity can be the uptake or amount of sugar, such as glucose or glycogen, or the amount of metabolite, such as lactate. In another embodiment, the feature is the tumor surface and the quantity can be roughness.

In one embodiment, the quantity of the second measurement is decreased in comparison to the first measurement. In such an embodiment, the patient has a cancer which is responsive to the compound (I), salt or pharmaceutical composition thereof. For example, in a responsive cancer, the quantity of GLUT4 in the second measurement is decreased in the second measurement compared to the first measurement. In another example of a responsive cancer, the quantity of tumor metabolic activity is decreased in the second measurement compared to the first measurement. In another example of a responsive cancer, the quantity of surface roughness is decreased in the second measurement compared to the first measurement. In an embodiment of a responsive cancer, the method comprises continuing the administration of the compound (I), salt or pharmaceutical composition thereof at the same dose, regimen or timing as in the first administration.

In another embodiment, the quantity of the second measurement is not decreased in comparison to the first measurement. In such an embodiment, the patient has a cancer which is not responsive to the dose of the compound (I), salt or pharmaceutical composition thereof which was administered prior to the second measurement. In one embodiment of a nonresponsive cancer, the method comprises the administration of the compound (I), salt or pharmaceutical composition thereof at more aggressive regimen, such as a higher dose, more frequent timing or by a route which increases bioavailability of the compound (I), salt or pharmaceutical composition thereof, compared to the first administration. In another embodiment of a nonresponsive cancer, the method comprises continuing the administration of the compound (I), salt or pharmaceutical composition thereof at the same dose, regimen or timing as in the first administration and also treating with a second therapeutic agent.

In one aspect, the present invention provides a method for treating cancer, comprising measuring a quantity of the metabolic activity of the cancer by a biomedical imaging technique, administering a therapeutically effective amount of the compound of formula (I):

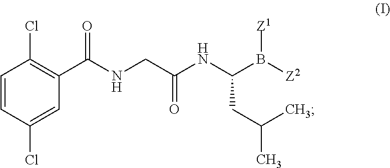

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient, repeating the measurement, and administering one of three types of treatment based on the relationship of the quantity in the second measurement to the quantity in the first measurement.

The treatment option can comprise: i) continuing to treat the cancer with the same dose of the compound if the quantity in the second measurement is lower than in the first measurement; ii) treating the cancer with a higher dose of the compound if the quantity in the second measurement is not lower than in the first measurement; and iii) continuing to treat the cancer with the same dose of the compound and a therapeutically effective amount of a second compound if the quantity in the second measurement is not lower than in the first measurement.

In some embodiments, the method for treating cancer comprises measuring a quantity of a feature of the cancer by a biomedical imaging technique, administering a therapeutically effective amount of the compound of formula (III-A):

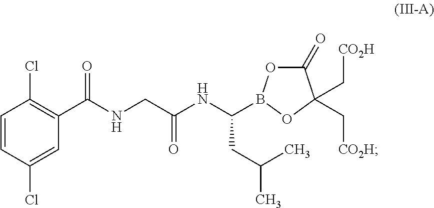 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof;
to the patient, repeating the measurement, and administering one of three types of treatment based on the relationship of the quantity in the second measurement to the quantity in the first measurement.

In one embodiment, the cancer comprises a solid tumor. In one embodiment, the solid tumor is from lung cancer. In another embodiment, the solid tumor is from colon cancer. In another embodiment, the cancer comprises a hematological tumor. In one embodiment, the hematological tumor is lymphoma, such as diffuse large cell lymphoma (DLCL). In another embodiment, the cancer comprises a tumor whose genotype comprises wild type KRAS (v-Ki-ras2 Kirsten rat sarcoma viral oncogene homolog). In one embodiment, the tumor has wild type KRAS or KRAS with mutated codon 146. In an embodiment, the tumor has wild type EGFR (epidermal growth factor receptor).

In an embodiment, the second measurement of the quantity is early after administering the compound of formula (III-A) or pharmaceutical composition thereof. In the embodiment, the time between administering the compound of formula (III-A) or pharmaceutical composition thereof and the second measurement is short. In some embodiments, the measurement is repeated again. In some embodiments, the additional repeat measurement or measurements are performed before another dose of the compound of formula (III-A) or pharmaceutical composition thereof. The additional repeat measurement can confirm the second measurement.

In one embodiment, the feature is metabolic capacity. In such an embodiment the quantity can be the amount of glucose transporter 4 (GLUT4) expression. In another embodiment, the feature is metabolic activity. In such an embodiment, the quantity can be the uptake or amount of sugar, such as glucose or glycogen, or the amount of metabolite, such as lactate. In another embodiment, the feature is the tumor surface and the quantity can be roughness.

In one embodiment, the quantity of the second measurement is decreased in comparison to the first measurement. In such an embodiment, the patient has a cancer which is responsive to the compound (III-A) or pharmaceutical composition thereof. For example, in a responsive cancer, the quantity of GLUT4 in the second measurement is decreased in the second measurement compared to the first measurement. In another example of a responsive cancer, the quantity of tumor metabolic activity is decreased in the second measurement compared to the first measurement. In another example of a responsive cancer, the quantity of surface roughness is decreased in the second measurement compared to the first measurement. In an embodiment of a responsive cancer, the method comprises continuing the administration of the compound (III-A) or pharmaceutical composition thereof at the same dose, regimen or timing as in the first administration.

In another embodiment, the quantity of the second measurement is not decreased in comparison to the first measurement. In such an embodiment, the patient has a cancer which is not responsive to the dose of the compound (III-A) or pharmaceutical composition thereof which was administered prior to the second measurement. In one embodiment of a nonresponsive cancer, the method comprises the administration of the compound (III-A) or pharmaceutical composition thereof at more aggressive regimen, such as a higher dose, more frequent timing or by a route which increases bioavailability of the compound (III-A) or pharmaceutical composition thereof, such as by intravenous administration, compared to the first administration. In another embodiment of a nonresponsive cancer, the method comprises continuing the administration of the compound (III-A) or pharmaceutical composition thereof at the same dose, regimen or timing as in the first administration and also treating with a second therapeutic agent.

In some embodiments, the method for treating cancer comprises measuring a quantity of the metabolic activity of the cancer by a biomedical imaging technique, administering a therapeutically effective amount of the compound of formula (III-A):

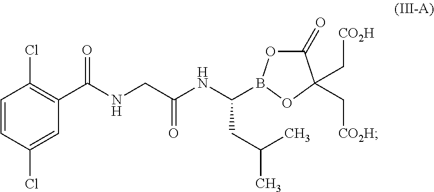 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof;
to the patient, repeating the measurement, and administering one of three types of treatment based on the relationship of the quantity in the second measurement to the quantity in the first measurement.

The treatment option can comprise: i) continuing to treat the cancer with the same dose of the compound if the quantity in the second measurement is lower than in the first measurement; ii) treating the cancer with a higher dose of the compound if the quantity in the second measurement is not lower than in the first measurement; and iii) continuing to treat the cancer with the same dose of the compound and a therapeutically effective amount of a second compound if the quantity in the second measurement is not lower than in the first measurement.

In one embodiment of the methods described above, the biomedical imaging technique is tomography. In another embodiment of the methods described above, the biomedical imaging technique is magnetic resonance. In another embodiment of the methods described above, the biomedical imaging technique is ultrasound.

In another aspect, the present invention provides a method for identifying a cancer patient who is nonresponsive to treatment with a compound of formula (I):

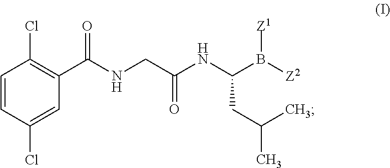

or a pharmaceutically acceptable salt or a pharmaceutical composition or a boronic acid anhydride thereof; wherein:
Z¹ and Z² are each independently hydroxy, alkoxy, aryloxy, or aralkoxy; or Z¹ and Z² together form a moiety derived from a boronic acid complexing agent;

comprising:
a) measuring the activity of the cancer by a biomedical imaging technique;
b) providing a therapeutically effective dose of the compound of formula (I) or a pharmaceutically acceptable salt or a pharmaceutical composition or a boronic acid anhydride thereof;
c) repeating the measurement of step a); and
d) identifying the patient as non-responsive to the compound if the cancer activity is not reduced.

In one aspect, the present invention provides a method for treating cancer comprising measuring a quantity of a feature of the cancer by a biomedical imaging technique, administering a therapeutically effective amount of the compound of formula (I):

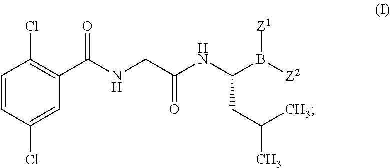

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient, repeating the measurement, and administering one of three types of treatment based on the relationship of the quantity in the second measurement to the quantity in the first measurement, i.e., whether the quantity has increased, e.g., whether the quantity in the second measurement is higher than in the first measurement.

In one embodiment, the feature is diffusivity, a measure of the ability of substances to flow through the tumor. In this embodiment, a responsive cancer becomes less dense, more diffuse, as a result of death of the tumor cells. In an embodiment, a biomedical imaging technique for measuring the diffusivity of cancer is diffusion weighted imaging. In an embodiment, an early increase in the diffusivity of the tumor can indicate responsiveness and thus, treatment should be continued.

In some embodiments, the invention provides a compound of anyone of formulas (I), (II), (III-A) or (III-B), or a pharmaceutically acceptable salt or a pharmaceutical composition or a boronic acid anhydride thereof, for use in treating cancer in a patient whose tumor has low metabolic activity, as measured in a biomedical imaging technique. In some embodiments, the invention provides a compound of anyone of formulas (I), (II), (III-A) or (III-B), or a pharmaceutically acceptable salt or a pharmaceutical composition or a boronic acid anhydride thereof, for use in treating cancer in a patient whose tumor decreases its metabolic activity early in the course of treatment, as measured in a biomedical imaging technique. In some embodiments, the invention provides a compound of anyone of formulas (I), (II), (III-A) or (III-B), or a pharmaceutically acceptable salt or a pharmaceutical composition or a boronic acid anhydride thereof, for use in treating cancer in a patient whose tumor has a low amount of GLUT4 expression, as measured in a biomedical imaging technique. In some embodiments, the invention provides a compound of anyone of formulas (I), (II), (III-A) or (III-B), or a pharmaceutically acceptable salt or a pharmaceutical composition or a boronic acid anhydride thereof, for use in treating cancer in a patient whose tumor decreases its GLUT4 expression early during the course of treatment, as measured in a biomedical imaging technique.

In some embodiments, the invention provides a compound of anyone of formulas (I), (II), (III-A) or (III-B), or a pharmaceutically acceptable salt or a pharmaceutical composition or a boronic acid anhydride thereof, for use in treating cancer in a patient comprising using a biomedical imaging technique to measure metabolic activity in the patient's tumor, determining if the patient's tumor has a low metabolic activity, and administering a therapeutically effective amount of the compound of anyone of formulas (I), (II), (III-A) or (III-B), or a pharmaceutically acceptable salt or a pharmaceutical composition or a boronic acid anhydride thereof, if the patient's tumor has a low metabolic activity. In some embodiments, the invention provides a compound of anyone of formulas (I), (III-A) or (III-B), or a pharmaceutically acceptable salt or a pharmaceutical composition or a boronic acid anhydride thereof, for use in treating cancer in a patient comprising using a biomedical imaging technique to measure metabolic activity in a tumor in a patient, administering a therapeutically effective amount of the compound of anyone of formulas (I), (II), (III-A) or (III-B), using the biomedical imaging technique to measure the tumor metabolic activity again, and proceeding with continued administration of the compound of anyone of formulas (I), (II), (III-A) or (III-B) or a pharmaceutically acceptable salt or a pharmaceutical composition or a boronic acid anhydride thereof, if the metabolic activity in the second measurement of the patient's tumor is decreased from the metabolic activity in the first measurement.

Unless otherwise defined, all technical and scientific terms used herein have the meanings which are commonly understood by one of ordinary skill in the art to which this invention belongs. Generally, nomenclature utilized in connection with, and techniques of cell and tissue culture, molecular biology and protein and oligo- or polynucleotide chemistry and hybridization described herein are those known in the art. GenBank or GenPept accession numbers and useful nucleic acid and peptide sequences can be found at the website maintained by the National Center for Biotechnology Information, Bethesda, Md. The content of all database accession records (e.g., from Affymetrix HG133 annotation files, Entrez, GenBank, RefSeq, COSMIC) cited throughout this application (including the Tables) are hereby incorporated by reference. Standard techniques are used for recombinant DNA, oligonucleotide synthesis, protein purification, tissue culture and transformation and transfection (e.g., electroporation, lipofection, etc). Enzymatic reactions, such as GTPase assay for RAS activity or assays, e.g., reporter assays, for RAS-activated signaling activity, are performed according to manufacturer's specifications or as commonly accomplished in the art or as described herein. Some methods for determining RAS localization and signaling are reviewed in Prior and Hancock (2011) Semin. Clin. Dev. Biol. September 8 epub; or found in Cuiffo and Ren (2010) Blood 114:3598-3605 or reviewed in Lim et al. (1996) Eur. J. Biochem. 242:171-185. The foregoing techniques and procedures generally are performed according to methods known in the art, e.g., as described in various general and more specific references that are cited and discussed throughout the present specification. See e.g., Sambrook et al. (2000) Molecular Cloning: A Laboratory Manual (3^{rd} ed., Cold Spring Harbor Laboratory Press, Cold Spring Harbor, N.Y.) or Harlow, E. and Lane, D. (1988) Antibodies: A Laboratory Manual (Cold Spring Harbor Laboratory Press, Cold Spring Harbor, N.Y.). The nomenclatures utilized in connection with, and the laboratory procedures and techniques of, analytical chemistry, synthetic organic chemistry, and medicinal and pharmaceutical chemistry described herein are known in the art. Standard techniques are used for chemical syntheses, chemical analyses, pharmaceutical preparation, formulation and delivery, and treatment of patients. Furthermore, unless otherwise required by context, singular terms shall include pluralities and plural terms shall include the singular. In the case of conflict, the present specification, including definitions, will control.

The articles “a,” “an” and “at least one” are used herein to refer to one or to more than one of the grammatical object of the article. By way of example, “an element” means one or more than one element, at least one element. In the case of conflict, the present specification, including definitions, will control.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

As used herein, the term “patient”, means an animal, such as a mammal, e.g., domesticated mammal or primate. For example, a patient is a human.

The present methods and compositions are designed for use in diagnostics and therapeutics for a patient suffering from cancer. A cancer or tumor is treated or diagnosed according to the present methods. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” further encompasses primary and metastatic cancers. Hematological tumors include tumors of hematological origin, including, e.g., myelomas (e.g., multiple myeloma), leukemias (e.g., Waldenstrom's syndrome, chronic lymphocytic leukemia, acute myelogenous leukemia, chronic myelogenous leukemia, other leukemias), lymphomas (e.g., B-cell lymphomas, non-Hodgkin's lymphoma) and myelodysplastic syndrome. Solid tumors can originate in organs, and include cancers such as in skin, lung, brain, breast, prostate, ovary, colon, kidney, pancreas, liver, esophagus, stomach, intestine, bladder, uterus, cervix, testis, adrenal gland, etc. The cancer can comprise a cell in which KRAS has a mutation. As used herein, cancer cells, including tumor cells, refer to cells that divide at an abnormal (increased) rate or whose control of growth or survival is different than for cells in the same tissue where the cancer cell arises or lives. Cancer cells include, but are not limited to, cells in carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large cell lymphoma, malignant lymphoma, plasmocytoma, reticulum cell sarcoma, or Hodgkins disease); and tumors of the nervous system including glioma, meningoma, medulloblastoma, schwannoma or epidymoma.

Unless otherwise explicitly stated, the term “proteasome” is intended to refer to constitutive proteasome, immunoproteasome, or both.

As used herein, the term “therapeutically effective amount” means an amount that is sufficient upon appropriate administration to a patient (a) to cause a detectable decrease in the severity of the disorder or disease state being treated; (b) to ameliorate or alleviate at least one of the patient's symptoms of the disease or disorder; or (c) to slow or prevent advancement of, or otherwise stabilize or prolong stabilization of, the disorder or disease state being treated (e.g., prevent additional tumor growth of a cancer). It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated.

Unless otherwise specified herein, the terms “antibody” and “antibodies” broadly encompass naturally-occurring forms of antibodies, e.g., polyclonal antibodies (e.g., IgG, IgA, IgM, IgE) and monoclonal and recombinant antibodies such as single-chain antibodies, two-chain and multi-chain proteins, chimeric, CDR-grafted, human and humanized antibodies and multi-specific antibodies, as well as fragments and derivatives of all of the foregoing, which fragments (e.g., dAbs, scFv, Fab, F(ab)′₂, Fab′) and derivatives have at least an antigenic binding site. Antibody derivatives may comprise a protein or chemical moiety conjugated to an antibody. The term “antibody” also includes synthetic and genetically engineered variants, such as monobodies and diabodies.

As used herein, “KRAS” refers to v-Ki-ras2 Kirsten rat sarcoma viral oncogene homolog, the gene associated with GenBank Accession No. NM_004985, SEQ ID NO:1 (open reading frame is SEQ ID NO:2, nucleotides 182 to 748 of SEQ ID NO:1), encoding GenPept Accession No. NP_004976, SEQ ID NO:3, the predominant transcript variant of KRAS gene on chromosome 12. Other names for KRAS include KRAS2, and Noonan Syndrome 3 (NS3). KRAS functions as an oncogene with GTPase activity and can be found on chromosome 12. KRAS interacts with the cell membrane and various effector proteins, such as Akt and Cdc42, which carry out its signaling function through the cytoskeleton and effects on cell motility (Fotiadou et al. (2007) Mol. Cel. Biol. 27:6742-6755). A mutated KRAS protein, e.g., an activating mutation, such as at residue 12 or 13 of SEQ ID NO:3, can prolong its time in the GTP-bound state and the resulting signaling pathway activation can lead to proliferation of cells harboring the mutated gene. Mutations in KRAS and their relationships to proteasome inhibition therapy, such as therapy with MLN9708, are described in PCT Publication No. WO2013071142, the contents of which are incorporated herein by reference.

As used herein, “EGFR” refers to epidermal growth factor receptor, the gene associated with GenBank Accession No. NM_005228, SEQ ID NO:7 (open reading frame is nucleotides 247 to 3879 of SEQ ID NO:7), encoding GenPept Accession No. NP_005219, SEQ ID NO:8, which has isoforms through splice variation. Other names for EGFR include ERBB and HER1. Binding of the epidermal growth factor to the receptor can cause tyrosine kinase signaling and cell proliferation.

Genes such as KRAS and EGFR are mutated in many cancer types. There has been interest in public cataloging of mutations associated with cancers. Examples of public databases which include information about mutations associated with cancers are the Database of Genotypes and Phenotypes (dbGaP) maintained by the National Center for Biotechnology Information (Bethesda, Md.) and Catalogue of Somatic Mutations in Cancer (COSMIC) database maintained by the Wellcome Trust Sanger Institute (Cambridge, UK).

As used herein, “GLUT4” refers to glucose transporter-4, the gene associated with GenBank Accession No. NM_001042, SEQ ID NO:4 (open reading frame is SEQ ID NO:5, nucleotides 201 to 1730 of SEQ ID NO:4), encoding GenPept Accession No. NP_001033, SEQ ID NO:6). Another name for GLUT4 is solute carrier family 2 (facilitated glucose transporter) member 4 (SLC2A4). GLUT4 functions as a glucose transporter and can be found on chromosome 17p. GLUT4 cellular location can depend on the presence of insulin, which stimulates cells such as muscle and adipose tissue to move GLUT4 from intracellular stores to the cell surface to commence its function as a glucose transporter. Glucose transporters, including GLUT1, GLUT4 and GLUT9 can have higher than normal activity in tumor cells to allow higher levels of glucose metabolism than in normal cells (reviewed Adekola et al. (2012) Curr. Opin. Oncol. 24:650-654). GLUT1, GLUT3 and GLUT4 can be expressed in lung carcinoma (Ito et al. (1999) Histol. Histopathol. 14:895-904). KRAS mutant colorectal cancer cells showed higher glucose uptake and glycolysis and better growth and survival under nutrient stress than wild type cells (Yun et al. 2009 Science 325:1555). Those studies identified a correlation between the upregulation of GLUT1, glucose transporter 1, with mutant KRAS in colorectal cancer cells, in contrast with an earlier study (Noguchi et al. (2000) Cancer Lett. 154:137-142).

As used herein, the term “noninvasive” refers to a procedure which inflicts minimal harm to a subject. In the case of clinical applications, a noninvasive sampling procedure can be performed quickly, e.g., in a walk-in setting, typically without anaesthesia and/or without surgical implements or suturing. Examples of noninvasive samples include blood, serum, saliva, urine, buccal swabs, throat cultures, stool samples and cervical smears. Noninvasive diagnostic analyses include x-rays, magnetic resonance imaging, positron emission tomography, etc.

A cancer is “responsive” to a therapeutic agent or there is a “good response” to a treatment if its rate of growth is inhibited as a result of contact with the therapeutic agent, compared to its growth in the absence of contact with the therapeutic agent. Growth of a cancer can be measured in a variety of ways, for instance, the characteristic, e.g., size of a tumor or the expression of tumor markers appropriate for that tumor type may be measured. International Working Groups convene periodically to set, update and publish response criteria for various types of cancers. Such published reports can be followed to support the identification of markers of the subject tumors and their response to proteasome inhibitors. For example, for solid tumors, the Response Evaluation Criteria in Solid Tumors (RECIST) guidelines (Eisenhauer et al. (2009) E. J. Canc. 45:228-247) can be used to support the identification of markers associated with solid tumors and response of solid tumors to a proteasome inhibitor. The response definitions used to support the identification of markers associated with myeloma and its response to an proteasome inhibitor, e.g., peptidyl boronic acid therapy, the Southwestern Oncology Group (SWOG) criteria as described in Blade et al. (1998) Br J Haematol. 102:1115-23 can be used. These criteria define the type of response measured in myeloma and also the characterization of time to disease progression which is another important measure of a tumor's sensitivity to a therapeutic agent. Other examples are criteria for Acute Myelogenous Leukemia (AML, Cheson et al. (2003) J. Clin. Oncol. 21:4642-4649), lymphomas, e.g., non-Hodgkin's and Hodgkin's lymphoma (Cheson et al. (2007) J. Clin. Oncol. 25:579-596). Criteria take into account analysis methods such as Positron Emission Tomography (PET), e.g., for identifying sites with measurable altered metabolic activity (e.g., at tumor sites) or to trace specific markers into tumors in vivo, immunohistochemistry, e.g., to identify tumor cells by detecting binding of antibodies to specific tumor markers, and flow cytometry, e.g., to characterize cell types by differential markers and fluorescent stains, in addition to traditional methods such as histology to identify cell composition (e.g., blast counts in a blood smear or a bone marrow biopsy, presence and number of mitotic figures) or tissue structure (e.g., disordered tissue architecture or cell infiltration of basement membrane). The quality of being responsive to a proteasome inhibitor, e.g., a peptidyl boronic acid therapy can be a variable one, with different cancers exhibiting different levels of “responsiveness” to a given therapeutic agent, under different conditions. Still further, measures of responsiveness can be assessed using additional criteria beyond growth size of a tumor, including patient quality of life, degree of metastases, etc. In addition, clinical prognostic markers and variables can be assessed (e.g., M protein in myeloma, PSA levels in prostate cancer) in applicable situations.

In some embodiments, a responsive tumor is characterized as having wild type KRAS. In some embodiments, a nonresponsive tumor is characterized as having mutated KRAS or activated KRAS. In some embodiments, an activating mutation is a mutation in codon 12, codon 13 or codon 61. A mutation at residue 146 of SEQ ID NO:3 is not activating. A responsive tumor can be from lung cancer, colon cancer, or diffuse large B-cell lymphoma. The identification of a mutation in KRAS can be made through the use of any of a number of techniques known to one skilled in the art, such as nucleic acid sequencing.

Biomedical imaging techniques are noninvasive methods to view features of an internal anatomical, physiological or biochemical feature, i.e. a mass, organ, tissue or cavity not exposed to the body surface. Such techniques include, but are not limited to, tomography, magnetic resonance and ultrasound. In some embodiments, the biomedical imaging technique is selected from the group consisting of computed tomography, magnetic resonance spectroscopy (MRS), magnetic resonance imaging (MRI), single photon emission computed tomography (SPECT), and positron emission tomography (PET).

Imaging techniques such as FDG-PET have become integrated in the standard-of-care for monitoring the treatment of certain cancers, but typically the monitoring evaluates the efficacy after at least one month or 1 full cycle of treatment or even six months or later after beginning treatment. As described herein, monitoring of treatment predicts responsiveness prior to treatment or evaluates the efficacy within the first cycle of treatment, in some embodiments as described below, just hours after beginning treatment.

In MRS and MRI, a patient lies in a superconducting magnet and a series of gradient and radio frequency pulses are applied to appropriately encode the contrast of the image acquisition to parameters associated with different physiological processes e.g. tissue necrosis, apoptotsis, proliferation, energy status, acidosis, vascular haemodynamics, and organ and/or lesion anatomy (see Hashemi R. H., Bradley W. G., & Lisanti C. J. MRI: The Basics. 2^{nd} Ed. 2004; Lippincott Williams and Wilkins). MRI and MRS can use ¹H, ¹³C or ³¹P imaging, and optionally, further can employ chelates of gadolinium or manganese for contrast. In some embodiments, MRS and MRI measure amounts of intracellular molecules to generate a metabolic profile. For example, glucose, lactate, choline, acetate, amino acids or ATP can be measured. In some embodiments, the MRI uses diffusion weighted imaging, which measures endogenous signals as a measure of tissue integrity. MRS can be used in a hyperpolarization mode, such as with ¹³C pyruvate, to increase the signal-to-noise ratio (Ardenkjaer-Larsen et al. (2003) Pros. Nat. Acad. Sci. U.S.A. 100:10158-10163, Bohndiek et al. Mol. Cancer Ther. (2010) 9:3278-3288).

A common parameter to measure with MRI, for example diffusion-weighted imaging is the Apparent Diffusion Coefficient (ADC). ADC increases in responders, due to an increase in diffusion as a result of decrease in cellularity related to tumor cell death. A responsive tumor ADC can increase 10%, 20%, 30%, 10 to 30%, 40%, 50%, 60%, 70%, 80% or 90% in the second measurement compared to the first.

A common parameter to measure with MRS is the total choline concentration (tCho) and ratios of endogenous amino acids. The tCho and amino acids can change 10%, 20%, 30%, 10 to 30%, 40%, 50%, 60%, 70%, 80% or 90% in the second measurement compared to the first in a responsive tumor.

In PET, a patient is injected with a radioactive substance that emits positrons, which can be monitored as the substance moves through the body, accumulates in target cell types or binds to known receptors. Examples of the radioactive substance used in PET include, but are not limited to, ¹⁸F-3′-deoxy-3′-fluoro-deoxyglucose, ¹⁸F-3′-deoxy-3′-fluorothymidine, and ¹⁸F-fluoromisonidazole (¹⁸F-MISO); which provide measures associated with cellular metabolism, proliferation, and hypoxia respectively (see Hendee W., Russell Ritenour E., Medical Imaging Physics 4^{th} Ed. 2004; Wiley Liss). In some embodiments, the radioactive substance used in PET is ¹⁸F-MISO. Additionally, imaging agents for PET can use positron emitters of oxygen, nitrogen, iron, carbon, or gallium.

PET can measure the uptake of glucose through the use of ¹⁸F-3′-deoxy-3′-fluoro-deoxyglucose (FDG). PET can measure proliferation of tumor cells through the use of ¹⁸F-3′-deoxy-3′-fluorothymidine (FLT) or ¹¹C-choline. PET can measure fatty acid synthesis through the use of ¹¹C-acetate.

A common parameter to measure with PET is the Standard Uptake Value (SUV). The SUV can be measured as SUVmax, SUVmean or SUVaverage (SUVave) or SUVpeak. Metabolically active tumors can have a SUVmax of about 30, about 30 to about 20 or about 25 to about 10. The SUV of the tumor can be compared to the SUV of a non-involved organ or tissue, such as liver or muscle. A ratio can be calculated (tumor-to-liver ratio (TLR) or tumor-to-muscle ratio (TMR), to determine whether there is an elevation of activity over normal tissue. Treatment can be beneficial if the metabolic activity of a patient's tumor is low. For example, low metabolic activity can be measured as a low SUV, such as be <8, <7, <6, <5, <4, <3, <2 or <1 for FDG or FLT. In some embodiments a low SUV is about 0.5 to about 1.5. In another example, low metabolic activity can be measured as a low TMR or TLR, such as <4, <3, <2 or <1.

The level of activity also can be compared with a scale derived from reference tumor measurements, such as historical measurements of tumors of the same type as found in the patient. In one embodiment, the level of activity can be compared to a baseline or basal measurement of a tumor, e.g., a tumor prior to therapy. In another embodiment, the pre-determined technique standard for comparison is the amount in the bladder reservoir.

The metabolic activity of the tumor can be measured before and after treatment to determine the treatment effect on metabolic activity. For example, a tumor which has an SUV which is >8, >7, >6, >5, >4, >3, >2 or >1 before treatment and <8, <7, <6, <5, <4, <3, <2 or <1, respectively (e.g., >8 before, <8 after, >7 before, <7 after, etc), after treatment can be a responsive tumor. A responsive tumor SUV can decrease 10%, 20%, 30%, 10 to 30%, 40%, 50%, 60%, 70%, 80% or 90% in the second measurement compared to the first. A responsive tumor SUV, e.g., SUVave, can decrease more than about 20%, more than about 25%, more than about 30%, more than about 40%, or more than about 50% in the second measurement compared to the first. A nonresponsive tumor SUV, e.g., SUVave, can be unchanged or change less than about 20% from baseline, e.g., change about −20% to about +20%, change less than about 15% from baseline, e.g., change about −15% to about +15%, or change less than about 10% from baseline, e.g., change about −10% to about +10%, in the second measurement compared to the first. A tumor which has TMR or TLR>4, >3, >2 or >1 before treatment and <4, <3, <2 or <1, respectively after treatment, can be a responsive tumor. A responsive tumor TMR or TLR can decrease 10%, 20%, 10 to 30%, 30%, 40%, 50%, 60%, 70%, 80% or 90% in the second measurement compared to the first.

The first measurement of a tumor, e.g., a baseline measurement, can be no earlier than 2 weeks, about 2 weeks, no earlier than 1 week, about 1 week, no earlier than 2 days, about 2 days, no earlier than 1 day, or about 1 day before therapy, such as a proteasome inhibitor therapy. The second measurement of a tumor can be 3 hours, 6 hours, 8 hours, 10 hours, 12 hours, 15 hours, 18 hours, 24 hours, 36 hours, 48 hours, or 72 hours after treatment with a therapy, such as a proteasome inhibitor therapy, e.g., after administration of a dose. The second measurement can be 1 to 8 days after beginning therapy or 5 to 10 days after beginning therapy. The second measurement can be no later than 4 days, no later than 1 week, no later than 10 days, no later than 2 weeks, no later than 3 weeks or no later than 1 month after therapy, e.g., after administration of a dose. The second measurement can be in the first cycle of therapy. The second measurement can be prior to the second cycle of therapy. The second measurement can be about 5 days, about 4 days, about 3 days, about 2 days, or about 1 day prior to the start of the second cycle of therapy. The second measurement can be after one dose of therapy but before the second dose. The second measurement can be after two doses of therapy but before the third dose. The second measurement can be after three doses of therapy but before the fourth dose. In an embodiment, the second measurement is 48 hours after the first dose of therapy. In another embodiment, the second measurement is 1 week after the first dose of therapy. For example, in FDG-PET, an early cytotoxic effect, e.g., with FDG-avid macrophages, can mask a metabolic shift by the tumor. In some embodiments, the second, post-treatment or endpoint FDG-PET measurement is performed after the cytotoxic activity but before or during the time that the drug would operate through the stress response and affect the metabolic activity of the tumor.

As used herein, a “cycle” of treatment with a compound, e.g., a proteasome inhibitor, described herein refers to one or more doses in the span of a number of days, in a repeating pattern. In one embodiment, a cycle is about 21 days in duration (a “21-day cycle”). In another embodiment, a cycle is about 28 days in duration (a “28-day cycle”). In one embodiment, a dosing regimen comprises a dose at day 1, 8, and 15 of a cycle. In another embodiment, a dosing regimen comprises a dose at day 1, 4, 8, and 11 of a cycle. In one embodiment, a dosing regimen comprises a dose at day 1, 8, and 15 of a 28-day cycle. In another embodiment, a dosing regimen comprises a dose at day 1, 4, 8, and 11 of a 21-day cycle.

In SPECT, a patient ingests or is injected with a radioactive substance that emits gamma radiation that can be detected by a gamma-camera as the substance moves through the body, accumulates in target cell types or binds to known receptors. Examples of the radioactive substance used in SPECT include, but are not limited to, ^{99m}Tc MDP/HDP, Sestamibi (Cardiolite®), ¹¹¹In-CYT-356 (ProstaScint) and ¹¹¹In-Zevalin (see Hendee W., Russell Ritenour E., Medical Imaging Physics 4^{th} Ed. 2004; Wiley Liss).

In some embodiments, the non-invasive technique comprises one or more of the different non-invasive techniques described above. In some embodiments, the non-invasive technique comprises PET and MRS. In some other embodiments, the non-invasive technique comprises PET and SPECT. In yet some other embodiments, the non-invasive technique comprises PET and MRI. In some further embodiments, the non-invasive technique comprises SPECT and MRS. In still yet some further embodiments, the non-invasive technique comprises SPECT and MRI.

In some embodiments, wherein the non-invasive technique comprises one or more of the different non-invasive techniques described above, each non-invasive technique is performed using a different machine or instrument.

In vivo imaging can be accomplished using known techniques and instructions from the manufacturer of the equipment employed for the analysis. The exact settings for the machine or instrument utilized for each non-invasive technique described above will depend on the specific instruments and machine. Those of skill in the art will be able to select such settings.

Biomedical imaging techniques which can measure metabolic activity of cancer include PET, SPECT and MRS. In some embodiments, metabolic activity of a cancer which is nonresponsive to proteasome inhibition therapy as measured by PET, SPECT or MRS, is high prior to treatment with a proteasome inhibitor.

Computed tomography (CT) can provide a view of the tumor surface and can provide images to measure roughness of the surface. The surface of a tumor which is responsive to proteasome inhibition therapy can have less roughness than a non-responsive tumor. Imaging by CT scan can employ a heavy metal such as iron chelate.

Examples of labels useful for diagnostic imaging in accordance with the present invention are radiolabels such as ₁₃₁I, ₁₁₁In, ₁₁₃In, ₆₇Ga, ₆₈Ga, ₁₂₃I, ₉₉ₘTc, ₃₂P, ₁₂₅I, ₁H, ₃H, ₁₄C, and ₁₈₈Rh, ₄₃K, ₅₂Fe, ₅₇Co, ₆₇Cu, ₇₇Br, ₈₁Rb/₈₁MKr, ₈₇Sr, ₁₂₇Cs, ₁₂₉Cs, ₁₃₂I, ₁₉₇Hg, ₂₀₃Pb, ₈₉Zr and ₂₀₆Bi, fluorescent labels such as fluorescein and rhodamine, nuclear magnetic resonance active labels, positron emitting isotopes detectable by a single photon emission computed tomography (“SPECT”) detector or positron emission tomography (“PET”) scanner, chemiluminescers such as luciferin, and enzymatic markers such as peroxidase or phosphatase. Short-range radiation emitters, such as isotopes detectable by short-range detector probes, such as a transrectal probe, can also be employed. Long-lived labels, such as ₈₉Zr, ₁₁₁In, ₄₄Sc, ₆₄Cu, ₈₆Y, ₁₂₄I, or ₁₅₂Tb, can allow repeat measurements, after one or more hour's time, e.g., 1 to 10 hours (e.g., 2 hours, 3 hours, 4 hours, 5 hours, 6 hours, 7 hours or 8 hours), to confirm the previous readings. Confirmation of measurements may be beneficial in situations where a non-responsive tumor may reserve metabolic capacity or machinery for metabolic activity in intracellular vesicles and an initial measurement may provide a false impression of a decreased metabolic capacity or metabolic activity.

Biomedical imaging techniques which can measure the metabolic capacity by quantifying transporter, e.g., GLUT4, expression include PET, SPECT, and ultrasound. The quantity of GLUT4 expression can be measured using an antibody which binds to GLUT4, such as an extracellular portion of GLUT4. Several GLUT4 antibodies are available, such as from Abcam (Cambridge, Mass.), R&D Systems (Minneapolis, Minn.) and Cell Signaling Technology, Inc. (Danvers, Mass.). Alternatively, an antibody which binds to GLUT4 or a portion thereof, such as an extracellular loop, can be generated by any means known to those skilled in the art. For example, for immunization, SEQ ID NO:6 can be expressed recombinantly as a full length polypeptide isolated in solution or integrated into the membrane of a mammalian cell or a portion or multiple portions can be synthesized chemically or displayed on a phage surface by methods known in the art. Computer programs and other research can identify the topology of GLUT4 and predict the locations of the extracellular loops useful for preparing immunogenic fragments of GLUT4. For example, valine at residue 383 of SEQ ID NO:6 is in an extracellular loop. Alternatively, expression of GLUT4 in a eukaryotic cell will orient the extracellular loops on the cell surface for immunogenic access. For immunization in a suitable (i.e., immunocompetent) subject such as a rabbit, goat, mouse, or other mammal or vertebrate, a composition comprising the GLUT4 polypeptide or portion thereof can further include an adjuvant, such as Freund's complete or incomplete adjuvant, or a similar immunostimulatory agent, such as keyhole limpet hemocyanin (KLH). (See generally, Harlow, E. and Lane, D. (1988) Antibodies: A Laboratory Manual, Cold Spring Harbor Laboratory Press, Cold Spring Harbor, N.Y.; and Current Protocols in Immunology, Coligan et al. ed., John Wiley & Sons, New York, 1994.) Monoclonal antibodies (see, e.g., Kohler and Milstein, Nature 256: 495 (1975)) made from the host response to immunization can be human through transgenic technology (see, e.g., XENOMOUSE™ technology, U.S. Pat. Nos. 6,162,963, 6,150,584, 6,114,598 and 6,075,181, which are incorporated herein by reference) or chimeric or humanized through recombinant technology (see, e.g., Reichmann, L. et al., Nature, 322: 323-327 (1988)). Human, humanized or chimeric antibodies can be less immunogenic in the patient than the natural host antibodies, so there can be less risk of an immune reaction to the imaging antibody upon repeat measurement of GLUT4 expression. In order not to harm the cells bound by the anti-GLUT4 imaging agent, mutations can be incorporated into the constant region (variant) of an antibody to GLUT4 to minimize binding to Fc receptors and/or ability to fix complement (see e.g. Winter et al, GB 2,209,757 B; Morrison et al., WO 89/07142; Morgan et al., WO 94/29351, Dec. 22, 1994).

Quantification of binding by an anti-GLUT4 antibody can be accomplished through direct or indirect labeling of the anti-GLUT4 antibody. For example, the label can be conjugated to the antibody or conjugated to a substance which binds the antibody, such as a secondary antibody or an enzyme complex. The antibody can be labeled with such reagents using techniques known in the art. For example, see Wensel and Meares (1983) Radioimmunoimaging and Radioimmunotherapy, Elsevier, New York, for techniques relating to the radiolabeling of antibodies. See also, D. Colcher et al. Meth. Enzymol. 121: 802-816 (1986). Antibody imaging can use known techniques (see e.g., A. R. Bradwell et al., Developments in Antibody Imaging”, Monoclonal Antibodies for Cancer Detection and Therapy, R. W. Baldwin et al., (eds.), pp 65-85 (Academic Press 1985)).

Suitable detectable substances include various biologically active enzymes, ligands, prosthetic groups, fluorescent materials, luminescent materials, chemiluminescent materials, bioluminescent materials, chromophoric materials, electron dense materials, paramagnetic (e.g., nuclear magnetic resonance active) materials, and radioactive materials. In some embodiments, the anti-GLUT4 antibody molecule is coupled to a radioactive ion, e.g., indium (₁₁₁In), iodine (₁₃₁I or ₁₂₅I), yttrium (₉₀Y), lutetium (₁₇₇Lu), actinium (₂₂₅Ac), bismuth (₂₁₂Bi or ₂₁₃Bi), sulfur (₃₅S), carbon (₁₄C), tritium (₃H), rhodium (₁₈₈Rh), technetium (₉₉mTc), praseodymium, or phosphorous (₃₂P); or a positron-emitting radionuclide, e.g., carbon-11 (₁₁C), potassium-40 (₄₀K), nitrogen-13 (₁₃N), oxygen-15 (₁₅O), fluorine-18 (₁₈F), zirconium-89 (₈₉Zr), and iodine-121 (₁₂₁I).

Exemplary labels include fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, and 2,3-dihydrophthalazinediones Other exemplary labels include horseradish peroxidase (HRP), alkaline phosphatase, galactosidase, glucoamylase, lysozyme, saccharide oxidases, e.g., glucose oxidase, galactose oxidase, and glucose 6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Fluorophore and chromophore labeled antibody molecules can be prepared from standard moieties known in the art. Since antibodies and other proteins absorb light having wavelengths up to about 310 nm, the fluorescent moieties should be selected to have substantial absorption at wavelengths above 310 nm and preferably above 400 nm. A variety of suitable fluorescent compounds and chromophores are described by Shyer Science, 162:526 (1968) and Brand, L. et al. Annual Review of Biochemistry, 41:843-868 (1972). The antibodies can be labeled with fluorescent chromophore groups by conventional procedures such as those disclosed in U.S. Pat. Nos. 3,940,475, 4,289,747, and 4,376,110.

In one embodiment, the invention provides a method for detecting the presence of GLUT4-expressing tumor tissues in vivo. The method includes (i) administering to a subject (e.g., a patient having a cancer) an anti-GLUT4 antibody, such as an antibody detected with a label or marker; (ii) exposing the subject to a means for detecting said label or marker to the GLUT4-expressing tissues or cells. In an embodiment to quantify GLUT4 expression by PET, an anti-GLUT4 antibody can use a zirconium label (e.g., ₈₉Zr). In an embodiment to quantify GLUT4 expression by SPECT, an anti-GLUT4 antibody can use an indium label (e.g., ₁₁₁In). In an embodiment to quantify GLUT4 expression by ultrasound (e.g., targeted contrast-enhanced ultrasound), an anti-GLUT4 antibody can use a microbubble label (see, e.g., Knowles et al (2012) Arch. Otolaryngol. Head Neck Surg. 137:662-668). Examples of tumors which can be analyzed using microbubble ultrasound include tumors in the breast, kidney and ovary.

The invention also includes kits for detecting the presence of GLUT4 in a patient. The kit can comprise a compound which binds to GLUT4, such as an anti-GLUT4 antibody. The compound or agent can be packaged in a suitable container. For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent or a means of coupling the first antibody to a radioactive tag for the imaging procedure.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used herein, the term “boronic acid” refers to a chemical compound containing a —B(OH)₂ moiety. In some embodiments, boronic acid compounds can form oligomeric anhydrides by dehydration of the boronic acid moiety. For example, Snyder et al., J. Am. Chem. Soc. 80:3611 (1958), reports oligomeric arylboronic acids.

As used herein, the term “boronic acid anhydride” refers to a chemical compound formed by combination of two or more molecules of a boronic acid compound, with loss of one or more water molecules. When mixed with water, the boronic acid anhydride compound is hydrated to release the free boronic acid compound. In various embodiments, the boronic acid anhydride can comprise two, three, four, or more boronic acid units, and can have a cyclic or linear configuration. Non-limiting examples of oligomeric boronic acid anhydrides of peptide boronic acids compound of the invention are illustrated below:

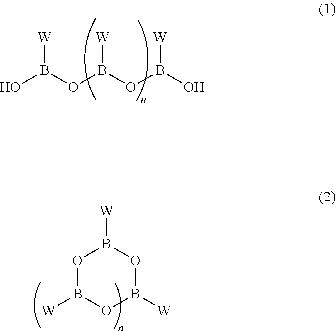

In formulae (1) and (2) directly above, the variable n is an integer from 0 to about 10, preferably 0, 1, 2, 3, or 4. In some embodiments, the boronic acid anhydride compound comprises a cyclic trimer (“boroxine”) of formula (2), wherein n is 1. The variable W has the formula (3):

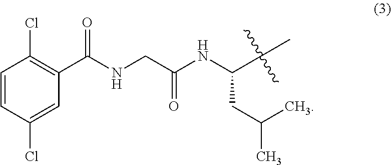 [SMILES: *[C@H](CC(C)C)NC(=O)CNC(=O)c1cc(Cl)ccc1Cl]

In some embodiments, at least 80% of the boronic acid present in the boronic acid anhydride compound exists in a single oligomeric anhydride form. In some embodiments, at least 85%, 90%, 95%, or 99% of the boronic acid present in the boronic acid anhydride compound exists in a single oligomeric anhydride form. In certain preferred embodiments, the boronic acid anhydride compound consists of, or consists essentially of, a boroxine having formula (3).

The boronic acid anhydride compound preferably can be prepared from the corresponding boronic acid by exposure to dehydrating conditions, including, but not limited to, recrystallization, lyophilization, exposure to heat, and/or exposure to a drying agent. Nonlimiting examples of suitable recrystallization solvents include ethyl acetate, dichloromethane, hexanes, ether, acetonitrile, ethanol, and mixtures thereof.

The term “alkyl”, used alone or as part of a larger moiety, refers to a straight or branched chain or cyclic aliphatic group having from 1 to 12 carbon atoms. The term “alkoxy” refers to an —O-alkyl radical.

The terms “aryl” and “ar-”, used alone or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is a C₆₋₁₀ aryl group. Aryl groups include, without limitation, phenyl, naphthyl, and anthracenyl. An “aralkyl” or “arylalkyl” group comprises an aryl group covalently attached to an alkyl group, either of which independently is optionally substituted. Preferably, the aralkyl group is C₆₋₁₀ aryl(C₁₋₆)alkyl, C₆₋₁₀ aryl(C₁₋₄)alkyl, or C₆₋₁₀ aryl(C₁₋₃)alkyl, including, without limitation, benzyl, phenethyl, and naphthylmethyl.

The term “substituted”, as used herein, means that a hydrogen radical of the designated moiety is replaced with the radical of a specified substituent, provided that the substitution results in a stable or chemically feasible compound. Nonlimiting examples of suitable substituents include C₁₋₆ alkyl, C₃₋₈ cycloalkyl, C₁₋₆alkyl(C₃₋₈)cycloalkyl, C₂₋₈ alkenyl, C₂₋₈ alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₆₋₁₀ aryl, C₁₋₆ alkyl(C₆₋₁₀)aryl, and halo(C₆₋₁₀)aryl.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

In some embodiments, Z¹ and Z² together form a moiety derived from a boronic acid complexing agent as disclosed in Olhava and Danca, U.S. Pat. Nos. 7,442,830, 7,867,662, and 8,003,819 all of which are herein incorporated by reference in their entirety. For purposes of the invention, the term “boronic acid complexing agent” refers to any compound having at least two functional groups, each of which can form a covalent bond with boron. Nonlimiting examples of suitable functional groups include amino, hydroxyl, and carboxyl. In some embodiments, at least one of the functional groups is a hydroxyl group. The term “moiety derived from a boronic acid complexing agent” refers to a moiety formed by removing the hydrogen atoms from two functional groups of a boronic acid complexing agent.

As used herein, the terms “boronate ester” and “boronic ester” are used interchangeably and refer to a chemical compound containing a —B(Z¹)(Z²) moiety, wherein at least one of Z¹ or Z² is alkoxy, aralkoxy, or aryloxy; or Z¹ and Z² together form a moiety derived from a boronic acid complexing agent having at least one hydroxyl group.

In some embodiments, Z¹ and Z² are each hydroxy and the compound of formula (I) is characterized by formula (II):

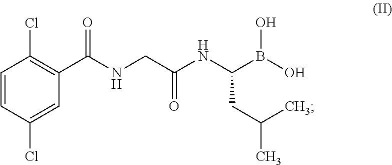 [SMILES: CC(C)C[C@H](NC(=O)CNC(=O)c1cc(Cl)ccc1Cl)B(O)O]

or a pharmaceutically acceptable salt or a pharmaceutical composition or a boronic acid anhydride thereof.

The compound of formula (II), [(1R)-1-({[(2,5-dichlorobenzoy)amino]acetyl}amino)-3-methylbutyl]boronic acid (MLN2238) is disclosed in Olhava and Danca, U.S. Pat. No. 7,442,830, herein incorporated by reference in its entirety.

In some other embodiments, Z¹ and Z² together form a moiety derived from a compound having at least two hydroxyl groups separated by at least two connecting atoms in a chain or ring, said chain or ring comprising carbon atoms and, optionally, a heteroatom or heteroatoms which can be N, S, or O, wherein the atom attached to boron in each case is an oxygen atom.

As employed herein, the term “compound having at least two hydroxyl groups” refers to any compound having two or more hydroxyl groups. For purposes of the invention, the two hydroxyl groups preferably are separated by at least two connecting atoms, preferably from about 2 to about 5 connecting atoms, more preferably 2 or 3 connecting atoms. For convenience, the term “dihydroxy compound” may be used to refer to a compound having at least two hydroxyl groups, as defined above. Thus, as employed herein, the term “dihydroxy compound” is not intended to be limited to compounds having only two hydroxyl groups. The moiety derived from a compound having at least two hydroxyl groups may be attached to boron by the oxygen atoms of any two of its hydroxyl groups. Preferably, the boron atom, the oxygen atoms attached to boron, and the atoms connecting the two oxygen atoms together form a 5- or 6-membered ring.

For purposes of the present invention, the boronic acid complexing agent preferably is pharmaceutically acceptable, i.e., suitable for administration to humans. In some embodiments, the boronic acid complexing agent is a sugar, as described, e.g., in Plamondon et al., WO 02/059131 and Gupta et al., WO 02/059130. The term “sugar” includes any polyhydroxy carbohydrate moiety, including monosaccharides, disaccharides, polysaccharides, sugar alcohols and amino sugars. In some embodiments, the sugar is a monosaccharide, disaccharide, sugar alcohol, or amino sugar. Non-limiting examples of suitable sugars include glucose, sucrose, fructose, trehalose, mannitol, sorbitol, glucosamine, and N-methylglucosamine. In certain embodiments, the sugar is mannitol or sorbitol. Thus, in the embodiments wherein the sugar is mannitol or sorbitol, Z¹ and Z² together form a moiety of formula C₆H₁₂O₆, wherein the oxygen atoms of the two deprotonated hydroxyl groups form covalent attachments with boron to form a boronate ester compound. In certain embodiments, Z¹ and Z² together form a moiety derived from D-mannitol as disclosed in U.S. Pat. No. 7,442,830, herein incorporated by reference in its entirety.

In some embodiments, the boronic acid complexing agent is an alpha-hydroxycarboxylic acid or a beta-hydroxycarboxylic acid, as described, e.g., in Elliott et al., WO 09/154737, herein incorporated by reference in its entirety. In some embodiments, the boronic acid complexing agent is selected from the group consisting of glycolic acid, malic acid, hexahydromandelic acid, citric acid, 2-hydroxyisobutyric acid, 3-hydroxybutyric acid, mandelic acid, lactic acid, 2-hydroxy-3,3-dimethylbutyric acid, 2-hydroxy-3-methylbutyric acid, 2-hydroxyisocaproic acid, beta-hydroxyisovaleric acid, salicylic acid, tartaric acid, benzilic acid, glucoheptonic acid, maltonic acid, lactobionic acid, galactaric acid, embonic acid, 1-hydroxy-2-naphthoic acid, and 3-hydroxy-2-naphthoic acid. In certain embodiments, the boronic acid complexing agent is citric acid.

In certain embodiments, wherein the alpha-hydroxy carboxylic acid or beta-hydroxy carboxylic acid is citric acid, the compound of formula (I) is characterized by formula (III-A) or (III-B):

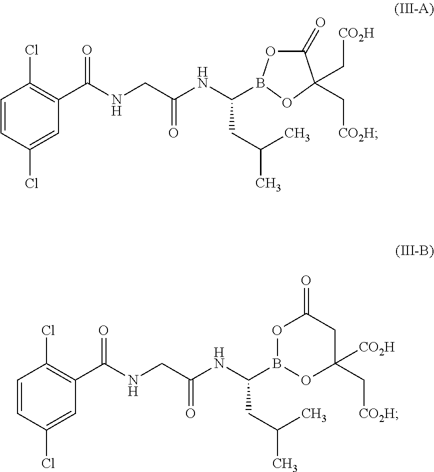 [SMILES: CC(C)C[C@H](NC(=O)CNC(=O)c1cc(Cl)ccc1Cl)B1OC(=O)C(CC(=O)O)(CC(=O)O)O1 ; CC(C)C[C@H](NC(=O)CNC(=O)c1cc(Cl)ccc1Cl)B1OC(=O)CC(CC(=O)O)(C(=O)O)O1]

or a mixture thereof or a pharmaceutical composition thereof.

In certain embodiments, wherein the alpha-hydroxy carboxylic acid or beta-hydroxy carboxylic acid is citric acid, the compound of formula (I) is characterized by formula (III-A):

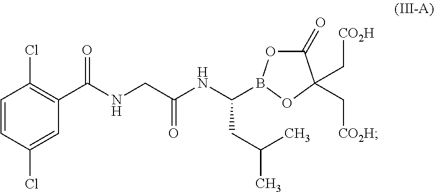 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof.

The compound of formula (III-A), 2,2′-{2-[(1R)-1-({[(2,5-dichlorobenzoy)amino]acetyl}amino)-3-methylbutyl]-5-oxo-1,3,2-dioxaborolane-4,4-diyl}diacetic acid (MLN9708) is disclosed in Elliott et al., WO 09/154737, herein incorporated by reference in its entirety.

In some embodiments, anyone of the compounds of formulas (I), (II), (III-A) or (III-B), or a pharmaceutically acceptable salt or a pharmaceutical composition or a boronic acid anhydride thereof is administered in conjunction with another therapeutic modality. In some embodiments, the compound of formula (III-A) or a pharmaceutical composition thereof is administered in conjunction with another therapeutic modality. In some embodiments, the therapeutic modality is one that is normally administered to patients with cancer. In some embodiments, the other therapeutic modality is radiotherapy. In some embodiments, the other therapeutic modality is another therapeutic agent. In some embodiments, the other therapeutic modality is radiotherapy and one or more therapeutic agents. The other therapeutic agent may be administered in the same dosage form or as a separate dosage form. When administered as a separate dosage form, the other therapeutic agent may be administered prior to, at the same time as, or following administration of anyone of the compounds of formulas (I), (II), (III-A) or (III-B), or a pharmaceutically acceptable salt or a pharmaceutical composition or a boronic acid anhydride thereof.

Non-limiting examples of therapeutic agents include DNA damaging chemotherapeutic agents which include topoisomerase I inhibitors (e.g., irinotecan, topotecan, camptothecin and analogs or metabolites thereof, and doxorubicin); topoisomerase II inhibitors (e.g., etoposide, teniposide, epirubicin, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Other non-limiting examples of therapeutic agents include chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate, erlotonib, sorafenib, crizitonib, vemurafenib and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, panitumumab, ipilimumab, and bevacizumab); and other inhibitors of proteins or enzymes known to be upregulated, over-expressed or activated in cancers, the inhibition of which downregulates cell replication.

In some embodiments, the therapeutic agent is selected from the group consisting of cisplatin, 5-flurouracil, epirubicin, docetaxel and paclitaxel.

Radiotherapy may be used as another therapeutic modality prior to, at the same time as, or following administration of anyone of the compounds of formulas (I), (II), (III-A) or (III-B), or a pharmaceutically acceptable salt or a pharmaceutical composition or a boronic acid anhydride thereof. In some embodiments, the radiotherapy is external beam radiotherapy. External beam radiotherapy is given as a series of treatments known as fractions. In some such embodiments, the external beam radiotherapy is conformal radiotherapy. In some embodiments, the radiotherapy is internal radiotherapy. Internal radiotherapy uses a radioactive substance sealed in needles, seeds, wires, or catheters that are placed directly into or near the cancer.

### Formulation and Administration

If a pharmaceutically acceptable salt of the compound of formula (I) is utilized in these compositions, the salt preferably is derived from an inorganic or organic acid or base. For reviews of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

Nonlimiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as lithium, sodium and potassium salts; alkaline earth metal salts, such as calcium and magnesium salts; other multivalent metal salts, such as zinc salts; salts with organic bases, such as dicyclohexylamine, N-methyl-D-glucamine, t-butylamine, ethylene diamine, ethanolamine, and choline; and salts with amino acids such as arginine, lysine, and so forth. In some embodiments, the pharmaceutically acceptable salt is a base addition salt of a boronic acid compound of formula (I), wherein Z¹ and Z² are both hydroxy.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, pH modifiers, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, carbonates, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, and mannitol, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer's solution, alcohols such as ethanol, isopropyl alcohol, hexadecyl alcohol, and glycerol, cyclodextrins such as hydroxypropyl β-cyclodextrin and sulfobutylether β-cyclodextrin, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

The pharmaceutical compositions utilized in the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions.

The pharmaceutical compositions utilized in the invention are formulated for pharmaceutical administration to a mammal, preferably a human being. Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intravenously, or subcutaneously. The formulations of the invention may be designed to be short-acting, fast-releasing, or long-acting. Still further, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

In some embodiments, the compound of formula (I) is administered orally. In some embodiments, the compound of formula (III-A) or a pharmaceutical composition thereof is administered orally. In some such embodiments, a pharmaceutical composition of the compound of formula (III-A) is prepared in gelatin capsules as described in Elliott et al., WO 09/154737, herein incorporated by reference in its entirety. In some embodiments, the pharmaceutical composition comprises the compound of formula (III-A) or a crystalline form thereof, a filler, optionally a lubricant, optionally a flow-aid and optionally a buffer. In some embodiments, the pharmaceutical composition comprises the compound of formula (III-A) or a crystalline form thereof, a filler, a lubricant, and a flow-aid. In some embodiments, the pharmaceutical composition comprises about 0.2% to about 12% of the compound of formula (III-A), or a crystalline form thereof, about 76.5% to about 99.8% of a filler, optionally up to about 1.5% of a lubricant, and optionally up to about 5% of a flow-aid. The oral pharmaceutical compositions can be prepared by methods described in Elliott et al., WO 09/154737, herein incorporated by reference in its entirety.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the compound of formula (I) is administered intravenously. In some such embodiments, the compound of formula (I) wherein Z¹ and Z² together form a moiety derived from a boronic acid complexing agent can be prepared in the form of a lyophilized powder, as described in Plamondon et al., WO 02/059131, herein incorporated by reference in its entirety or Elliott et al., WO 09/154737, herein incorporated by reference in its entirety. In some embodiments, the lyophilized powder also comprises free boronic acid complexing agent. Preferably, the free boronic acid complexing agent and the compound of formula (I) are present in the mixture in a molar ratio ranging from about 0.5:1 to about 100:1, more preferably from about 5:1 to about 100:1. In various embodiments, the lyophilized powder comprises free boronic acid complexing agent and the corresponding boronate ester in a molar ratio ranging from about 10:1 to about 100:1, from about 20:1 to about 100:1, or from about 40:1 to about 100:1.

In some embodiments, the lyophilized powder comprises boronic acid complexing agent and a compound of formula (I), substantially free of other components. However, the composition can further comprise one or more other pharmaceutically acceptable excipients, carriers, diluents, fillers, salts, buffers, bulking agents, stabilizers, solubilizers, and other materials well known in the art. The preparation of pharmaceutically acceptable formulations containing these materials is described in, e.g., Remington: The Science and Practice of Pharmacy, 20th Ed., ed. A. Gennaro, Lippincott Williams & Wilkins, 2000, or latest edition. In some embodiments, the pharmaceutical composition comprises a compound of formula (I), a bulking agent, and a buffer. In some embodiments, the pharmaceutical composition comprises a compound of formula (III-A), a bulking agent, and a buffer.

The lyophilized powder comprising the compound of formula (I) or formula (III-A) can be prepared according to the methods described in Plamondon et al., WO 02/059131, herein incorporated by reference in its entirety or Elliott et al., WO 09/154737, herein incorporated by reference in its entirety In some embodiments, the method for preparing the lyophilized powder comprises: (a) preparing an aqueous mixture comprising a boronic acid compound of formula (I), wherein Z¹ and Z² are each hydroxy, and a boronic acid complexing agent; and (b) lyophilizing the mixture. In some embodiments, the method for preparing the lyophilized powder comprises: (a) preparing an aqueous mixture comprising the compound of formula (III-A), a bulking agent, and a buffer; and (b) lyophilizing the mixture.

The lyophilized powder preferably is reconstituted by adding an aqueous solvent suitable for pharmaceutical administrations. Examples of suitable reconstitution solvents include, without limitation, water, saline, and phosphate buffered saline (PBS). Preferably, the lyophilized powder is reconstituted with normal (0.9%) saline. Upon reconstitution, an equilibrium is established between a boronate ester compound and the corresponding free boronic acid compound. In some embodiments, equilibrium is reached quickly, e.g., within 10-15 minutes, after the addition of aqueous medium. The relative concentrations of boronate ester and boronic acid present at equilibrium is dependent upon parameters such as, e.g., the pH of the solution, temperature, the nature of the boronic acid complexing agent, and the ratio of boronic acid complexing agent to boronate ester compound present in the lyophilized powder.

The pharmaceutical compositions utilized in the present invention preferably are formulated for administration to a patient having, or at risk of developing or experiencing a recurrence of cancer. Preferred pharmaceutical compositions utilized in the present invention are those formulated for oral, intravenous, or subcutaneous administration. Any of the above dosage forms containing a therapeutically effective amount of a compound of formula (I) are well within the bounds of routine experimentation and within the scope of the present invention. In some embodiments, the pharmaceutical composition utilized in the present invention may further comprise another therapeutic agent.

The amount of additional therapeutic agent present in a composition of this invention typically will be no more than the amount that would normally be administered in a composition comprising that therapeutic agent as the only active agent. Preferably, the amount of additional therapeutic agent will range from about 50% to about 100% of the amount normally present in a composition comprising that agent as the only therapeutically active agent.

In order that this invention be more fully understood, the following preparative and testing examples are set forth. These examples illustrate how to make or test specific compounds, and are not to be construed as limiting the scope of the invention in any way.

### EXAMPLES

### Example 1

### Preparation of Compounds and Pharmaceutical Compositions

The compound of formula (II), [(1R)-1-({[(2,5-dichlorobenzoy)amino]acetyl}amino)-3-methylbutyl]boronic acid, is prepared by methods disclosed in Olhava and Danca, U.S. Pat. No. 7,442,830, herein incorporated by reference in its entirety. The compound of formula (III-A), 2,2′-{2-[(1R)-1-({[(2,5-dichlorobenzoy)amino]acetyl}amino)-3-methylbutyl]-5-oxo-1,3,2-dioxaborolane-4,4-diyl}diacetic acid, is prepared by methods disclosed in Elliott et al., WO 09/154737, herein incorporated by reference in its entirety. An oral capsule formulation of the compound of formula (III-A) is prepared by methods disclosed in Elliott et al., WO 09/154737, herein incorporated by reference in its entirety. An IV formulation of the compound of formula (III-A) is prepared by methods disclosed in Elliott et al., WO 09/154737, herein incorporated by reference in its entirety. A lyophilized formulation of the compound of formula (III-A) suitable for reconstitution into an IV formulation is prepared by methods disclosed in Elliott et al., WO 09/154737, herein incorporated by reference in its entirety.

### Example 2

### FDG-PET Analysis of Primary Human Tumor Xenografts

The investigational agent MLN9708 is a citrate ester which immediately hydrolyzes in aqueous solution to MLN2238, the biologically active form (Kupperman et al (2010) Cancer Res. 70:1970-1980). MLN2238 was used as a surrogate for MLN9708 in the in vivo and in vitro studies described herein.

PHTX192Lu and PHTX132Lu primary human lung adenocarcinoma explants were grafted s.c. in SCID mice and grown to 200-300 mm3, randomized and selected for imaging. Two separate studies were performed on PHTX132Lu implanted animals to confirm reproducibility and robustness of FDG signal change (n=4-8/group). For each tumor line, three separate groups of animals were studied at different timepoints after treatment with MLN2238. Thus, all animals were scanned at baseline and then received MLN2238 (11 mg/kg, i.v.); three groups of animals were then scanned at 6, 24 and 48 hrs post dose Animals were fasted ˜6 hrs prior to imaging and received FDG administration 1 hour prior to imaging session. A 5 min static image (followed by an 8 minute attenuation correction scan) was acquired using a Siemens Inveon PET/CT (Siemens Medical, Knoxville, Tenn., USA). PET data was reconstructed using a 2D ordered-subset expectation maximization (OSEM) method, resulting in whole body images 128×128×63 voxels, using Inveon Acquisition Workshop (Siemens). Regions of interest were manually drawn over the whole tumor and standardized uptake values (SUV) maximum and average were extracted

$$( SUV = \frac{FDGregion}{FDGdose / Body Weight} ) .$$

All data were analyzed as a pairwise Student's t-test (where p<0.05 is significant) unless stated otherwise.

Earlier studies in immunocompromised mice have demonstrated the chronic 21 day anti-tumor activity of MLN2238 (11 mg/kg BIW, i.v.) in PHTX192Lu and PHTX132Lu primary human tumor explants, with moderate/low and high efficacy respectively (t/c average tumor volume treated÷average tumor volume control at day 21 to be 0.8 vs. 0.29).

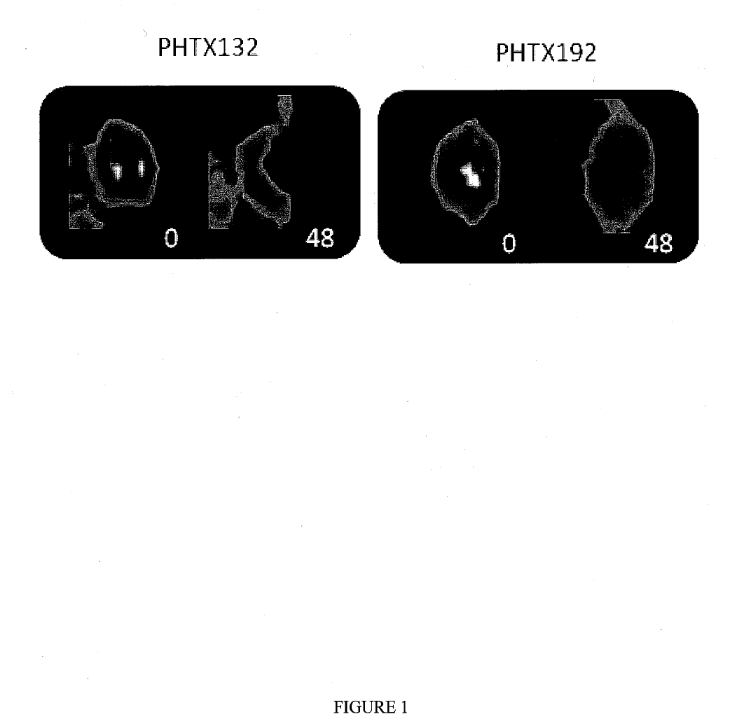

Study 1 (S1). FDG SUVmx from the PHTX132Lu demonstrated a significant 48 hr˜32% decrease. The FDG SUVave from the same study showed a significant ˜40% decrease. Qualitatively, it is noted that there is a near global reduction in the FDG signal in the tumor 48 hrs after treatment, yet the periphery still appears to be not as largely affected as the core. In contrast, the PHTX192Lu FDG signal in the tumor does not appear to have been greatly affected (FIG. 1).

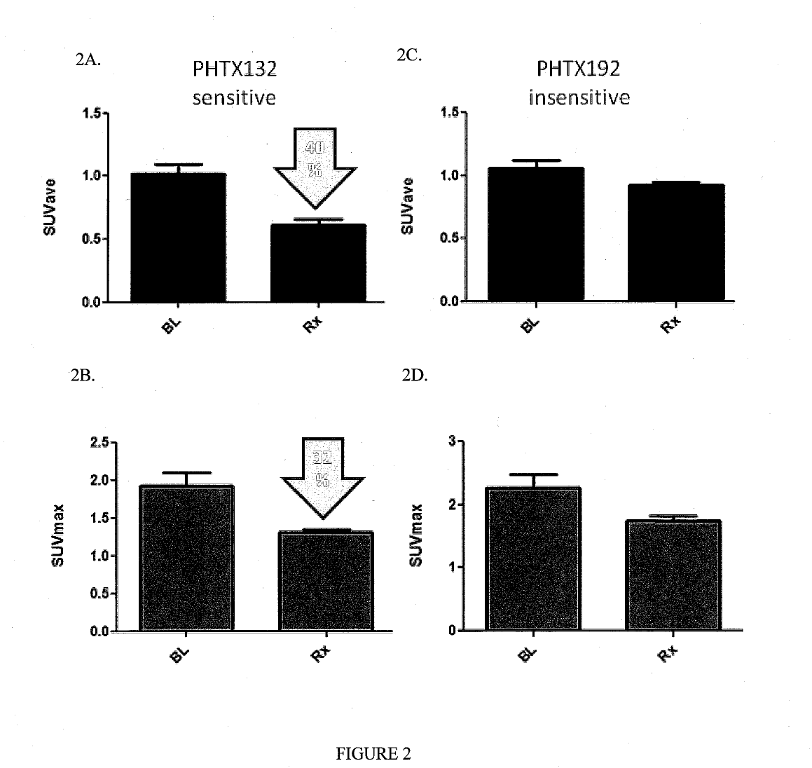

Study 2 (S2). A repeat study was performed in the PHTX132Lu. Decreases were observed in both FDG SUVmax (˜22%) and SUVave (˜34%) (FIGS. 2A and 2B), yet only the latter reached statistical significance.

To note; the basal FDG SUVave and FDG SUVmax for S1 and S2 were significantly different (SUVave 1.01 vs. 0.64 and SUVmax 1.92 vs. 1.18).

Study 3 (S3). In the PHTX192Lu unresponsive model, neither FDG SUVmax (˜23%) or SUVave (˜12%) (FIGS. 2C and 2D) showed significant changes 48 hrs after treatment.

FIGS. 2A-D (SUVave and SUVmax mean values at Time 0 and 48 hrs) and Table 1 summarize these observations. Interestingly, FDG SUVmax at 0 hrs was significantly higher in the PHTX192 vs. PHTX132 (S2) (student t-test unpaired, p<0.05) but only marginally higher than PHTX132 (S1). This basal glycolytic phenotype may play a part in the extent of the metabolic responsiveness to treatment with MLN2238 in sensitive models.

TABLE 1
SUV measurements of Primary Tumor Xenografts before and 48 h after MLN2238
treatment.

| | 0 hr | | 48 hr | | %age change from baseline | |
|---|---|---|---|---|---|---|
| | SUVmax | SUVave | SUVmax | SUVave | SUVmax | SUVave |
| PHTX132Lu | 1.92 | 1.01 | 1.3 | 0.61 | −32* | −40* |
| | (0.18) | (0.07) | (0.04) | (0.04) | [−10 to −56%] | [−8 to −65] |
| PHTX132Lu | 1.18 | 0.64 | 0.92 | 0.42 | −22 | −33* |
| (S2) | (0.1) | (0.06) | (0.16) | (0.08) | [49 to −76] | [7 to −66] |
| PHTX192Lu | 2.27 | 1.07 | 1.74 | 0.93 | −23 | −12 |
| | (0.21) | (0.05) | (0.08) | (0.02) | [−1.6 to −22] | [1.6 to −40] |

Standard deviations in parentheses ( ); range in brackets [ ]

In another study, 1) MLN2238 produced chronic anti tumor activity, as determined by conventional caliper tumor volume measurements, in the PHTX132Lu xenograft, 2) a single dose of MLN2238 produced a profound decrease in FDG signal in a tumor model that is chronically sensitive to MLN2238 and 3) a single dose of MLN2238 in a chronically insensitive model showed no significant change in FDG at 48 hrs. These data support the notion that an early FDG-PET response may predict for more chronic anti-tumor activity. Repeating studies in s.c. xenografts (S1 and S2) enabled reproducibility to be evaluated. Although a significant change was observed, there was diversity amongst the responsive group in two studies.

### Example 3

### More FDG-PET Studies of Tumor Xenografts with MLN2238 Treatment

Mice were implanted with tumors, treated MLN9708 (11 mg/kg, i.v.), fasted and imaged as in Example 2.

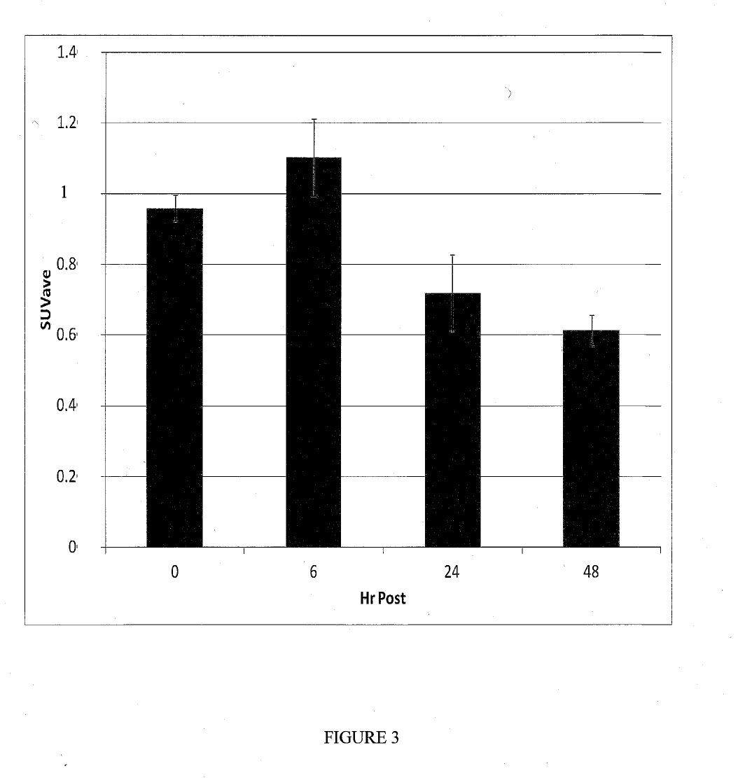

A. Further experiments were undertaken to determine FDG uptake between tumors (KRas vs WT) post MLN9708 treatment over acute time points (6, 24, & 48 hours). For the first study, an acute PD imaging study was undertaken with the primary tumor PHTX-132LU (KRas WT). MLN2238 (11 mg/kg, IV, N=7/group) was administered one time as a single bolus and the animals were imaged at their respective time points (0, 6, 24, & 48 hours). Each group received a baseline scan for ¹⁸F-FDG uptake 24 hours prior to MLN9708 administration. Quantitative analysis of the data indicated that SUVₘₐₓ and SUVₐᵥₑ showed a trend towards increased signal intensity at 6 hr with a significant decrease at 48 hours (FIG. 3, p=0.015). This is a responsive tumor, with treated/control of ˜0.30. The 6 hr time point showed a trend towards a “flare like” event in both parameters of SUVave and SUVmax. This was not noted as an unusual event as this phenomenon has been observed in other ubiquitin/proteasome pathway inhibitor projects such as MLN4924. Comparison of FDG uptake images at later time points finds a dramatic decrease in signal intensity of the tumor 24 and 48 h post MLN9708 administration.

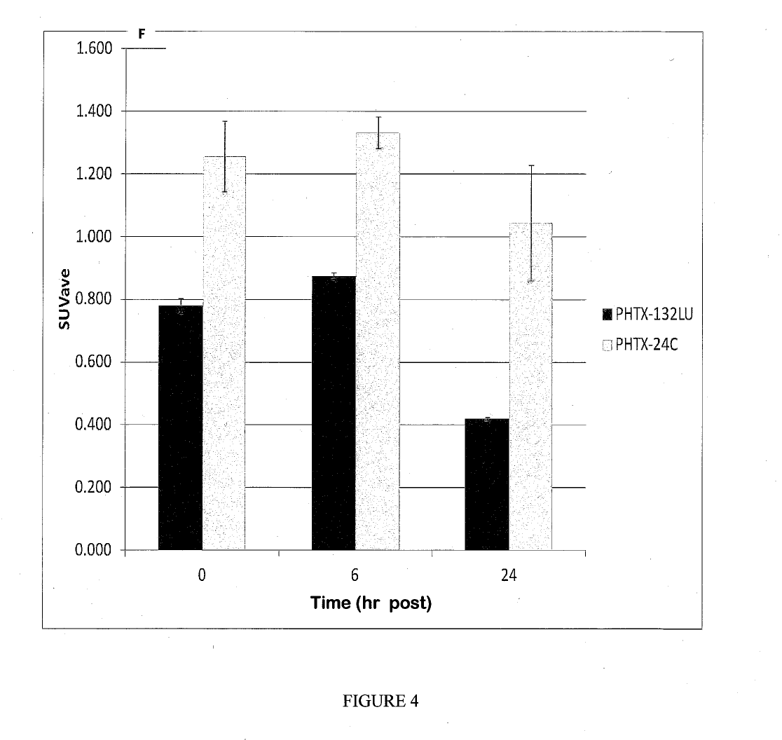

B. In another experiment (FIG. 4), the human primary tumor lines PHTX-24C and PHTX-132LU were utilized. The PHTS-24C (primary human colon) tumor has a rare A146T KRAS mutation. This mutation is not activating and the tumor acts like a wild type tumor. The limited PHTX-24C animal number (N=2/group/tumor line) provided only three time points (Baseline, 6, and 24 hr post MLN2238). On FDG-PET, its activity decrease at 24 h after treatment (MLN2238, 11 mg/kg IV) was qualitatively similar to the activity decrease of the PHTX-132Lu (lung tumor). Tumors at the 24 hr time period post MLN9708 treatment showed a decrease in FDG uptake for both tumor types (FIG. 4).

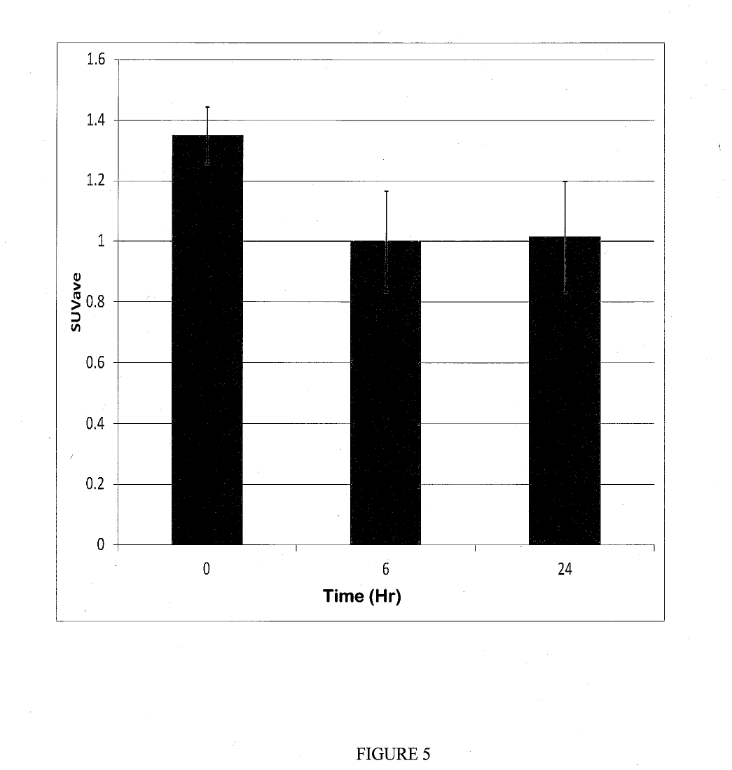

C. An imaging study with the primary tumor line PHTX-192LU (KRAS mutant) also was undertaken. This experiment only looked at two time points beyond baseline (6 and 24 hr) after MLN2238 treatment (11 mg/kg IV, treated/control ˜0.8). The number of animals used during the study was with an n=7 for the 6 hr group and n=6 for the 24 hr group. The data (FIG. 5) shows a slight decrease in SUVₐᵥₑ but signal intensity is stabilized over the 6-24 hr time point. There was no significance between the groups tested for this experiment.

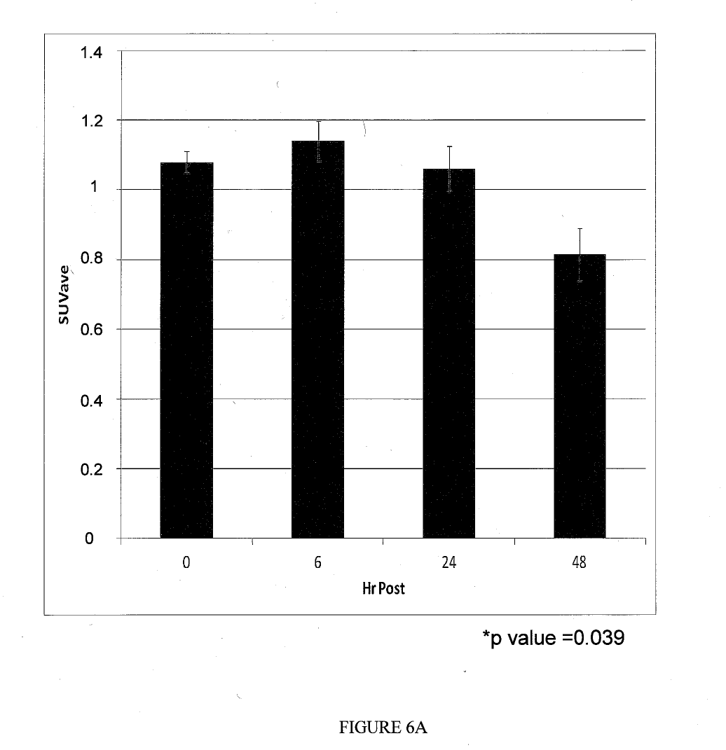

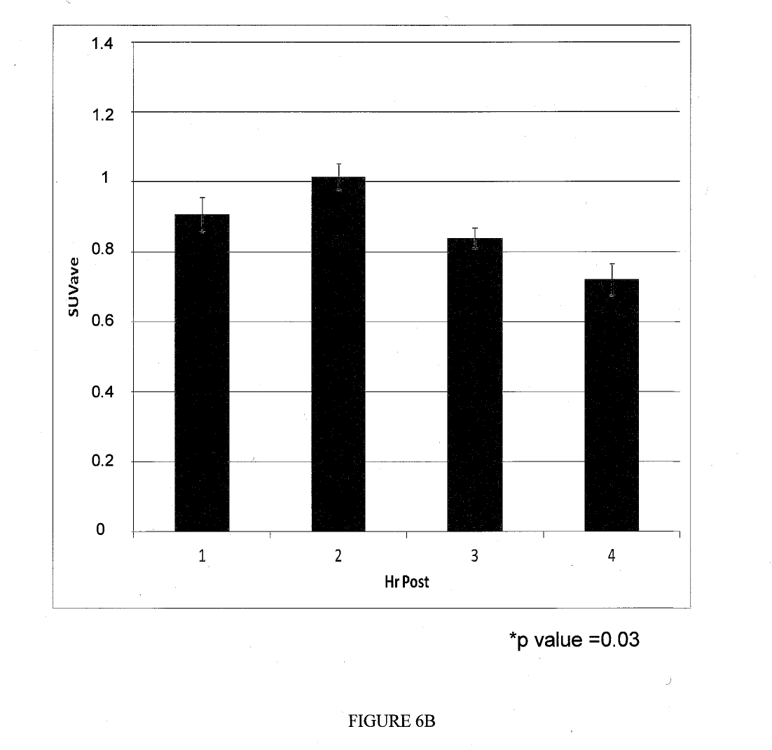

D. The Horizon isogenic cell line (SW48 WT KRas and SW48 KRas G13D) was tested in two different studies: (SW48, FIG. 6A) and (SW48 G13D, FIG. 6B) implanted as xenografts were performed as per the previous studies (MLN2238 11 mg/kg, IV, N=8) with baseline scans and FDG PET imaging at 6, 24, and 48 hours post MLN9708 administration. Unfortunately, the experiment did not follow the trend that had been seen with the clinical tumor lines previously tested. There was a slight difference between the two cell lines with FDG elevations at 6 hr above base line with the G13D tumors verses little change with the parental. Noted in this study was that the G13D line appeared to have stabilized FDG signal at the 24 and 48 hr period compared to the parental cell line which had a significant decrease in signal at the 48 hr period.

Based on data compiled from these experiments, the original hypothesis was redrawn. The KRAS mutation was theorized to provide a survival advantage to cancer by helping it cope with cellular stress. Further imaging analyses were devised to determine if this is a real event in which could be imaged with FDG and PET.

### Example 4

### Organotypic Cell Culture Imaging

A three-dimensional (3D) organotypic cell culture (OTOCs) technique was devised for the purpose of monitoring radio-labeled compound uptake in vitro. OTOC's are 3D cultures of tumor cells intermixed with cellular stromal matrix and formed into a sphere or a semisolid oval of about 2-3 mm thickness and about 3-7 mm in diameter. After culturing to establish the tumor-like 3D properties in the population of cells, they are incubated in media with 75 μCi ₁₈F-FDG for 1 h prior to imaging. This process provides a 3D environment, such as a nutrient diffusion gradient, more indicative of what is encountered in an in vivo setting than cultures grown on a plastic surface. In vitro experiments with the OTOC's suggested a difference in glucose uptake/utilization between the KRAS mutant and the WT cell line. The first study consisted of a 6 well plate with 6 OTOCs/well. The in vitro dosage of MLN9708 was extrapolated from historical in vivo PK studies for this compound. The 700 ng/ml concentration decided upon was derived from the mean tumor concentration achievable with a standard in vivo dose of MLN9708 (11 mg/kg PK dose, N).

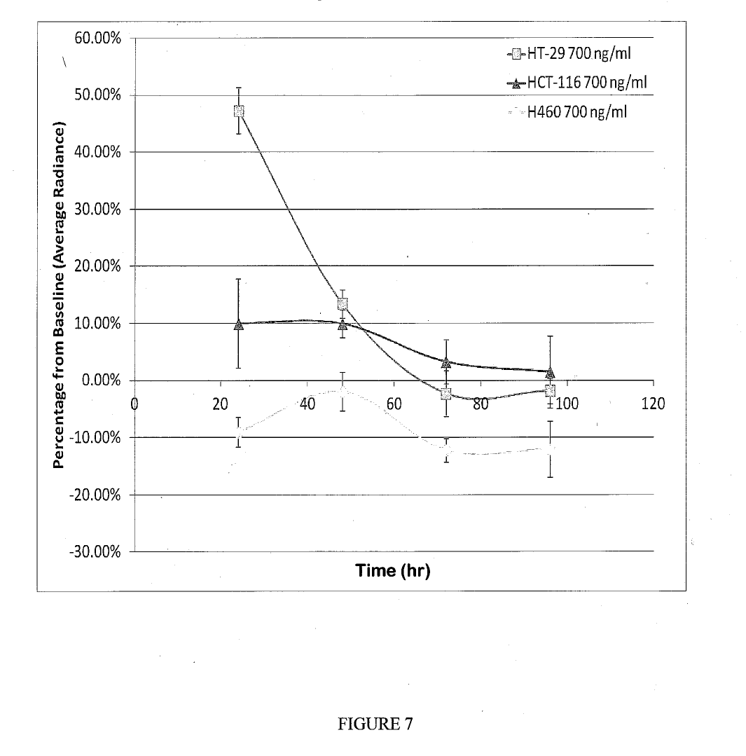

Three cell lines (HT-29, HCT-116, and H460) were used in the first OTOCs experiment to test the effects of FDG uptake after drug treatment. The imaging modality Cerenkov Luminescence Imaging (CLI, Robertson et al. (2009) Phys. Med. Biol. 54:N355-N365) was used to monitor the FDG response in vitro. The cell line HT-29 (KRAS WT) is responsive to MLN9708 in vivo (t/c<0.3), while HCT-116 and H460 (both KRAS mutants) have resistance to proteasome inhibition. OTOCs were imaged at 24, 48, 72, 96 hours post compound administration. The data in FIG. 7 shows a difference in FDG uptake values between the responsive and non responsive lines. HT-29 had an increased uptake at the 24 hr time point (48%) while the other two cell lines showed a marginal response (<10%) or a decrease in FDG signal (H460-10% decrease from control) at the early time point.

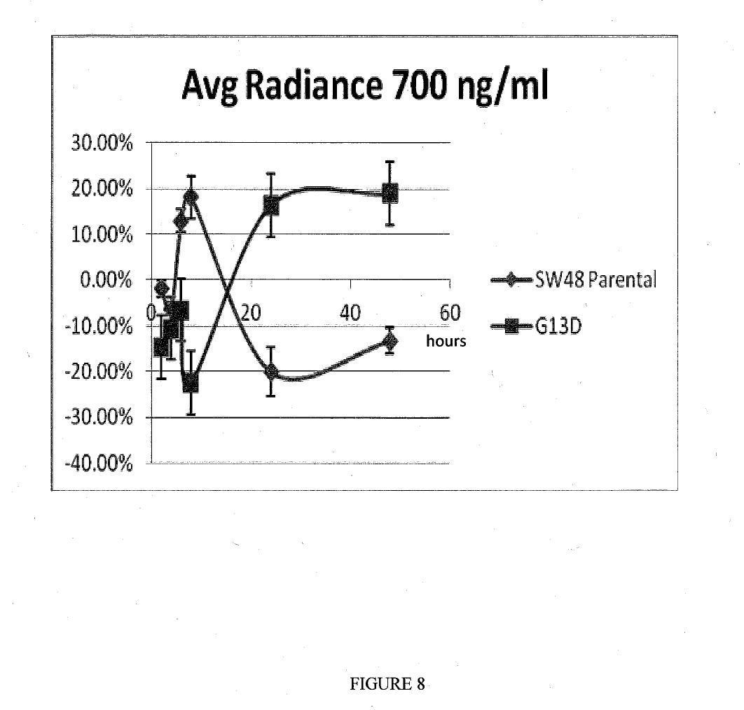

An OTOC's experiment was then conducted with the isogenic line SW48 and SW48 G13D (FIG. 8) at the 700 ng/ml concentration, with monitoring 18F-FDG uptake over time. The KRAS mutant behaved markedly different than those of the WT. The KRAS mutant had lower FDG uptake within an 8 hour period where the WT showed an increase in FDG uptake within the same 8 hour period. FIG. 8 shows the data plotted as normalized to control for average radiance over 60 h of monitoring.

### Example 5

### Further Xenograft Studies

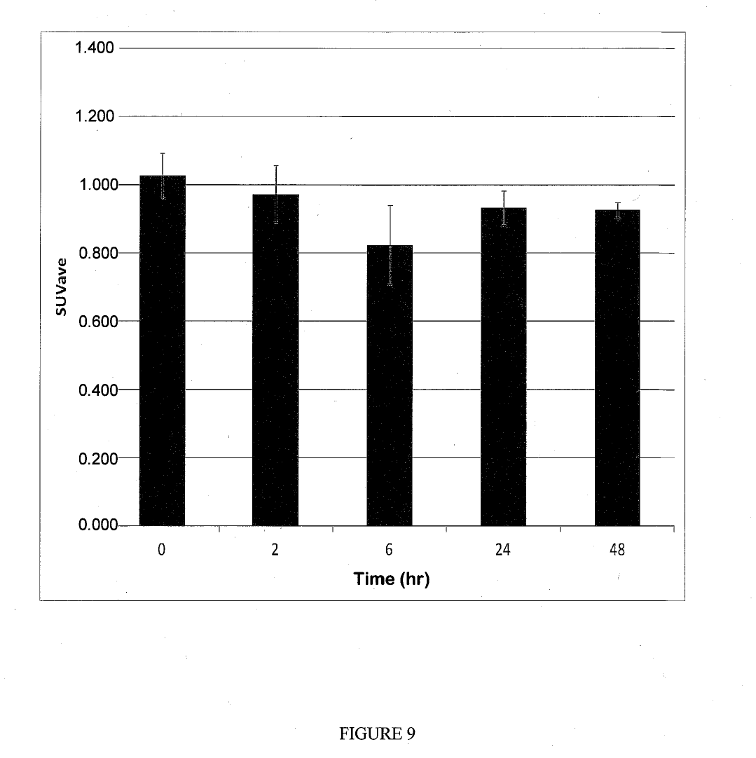

Based on the OTOCs data that was generated several additional in vivo studies were undertaken to determine if there was an early event that may have been missed in the original studies. A more extensive PD study with the KRAS mutant PHTX-192LU (FIG. 9) was run with more time points after MLN2238 treatment (11 mg/kg IV): baseline, 2, 6, 24, and 48 hr post drug administration (N=7 per group, treated/control was ˜0.8) to determine if the hypothesis was correct. As one can see from the data, the Kras mutant cell line appears to have an early event where FDG uptake has been diminished in the earlier time points and a stabilization/increase in FDG uptake at the later time points.

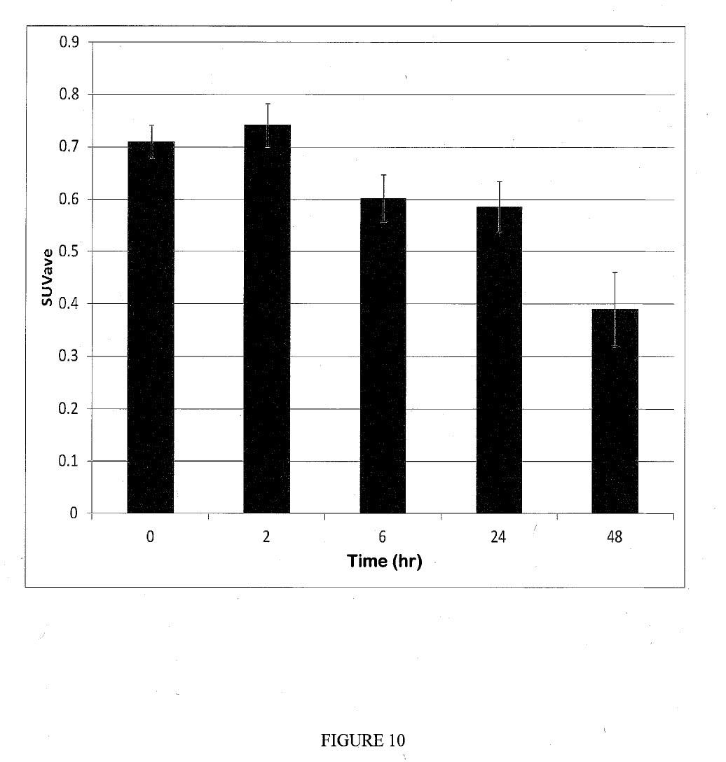

To reconfirm the orignal findings with the PHTX-132LU cell line (FIG. 10), a large study (n=8) with MLN2238 (11 mg/kg IV) treatment was run at a more extensive time frame due to the data acqured from the in vitro experiments. There appeared to be an increase in the earler time point with this KRAS mutant and a decrease in signal during progression to the later time points (difference in SUVave from 0-48 h timepoint has p-value of 0.038, treated/control was ˜0.30), reconfirming the orignal data.

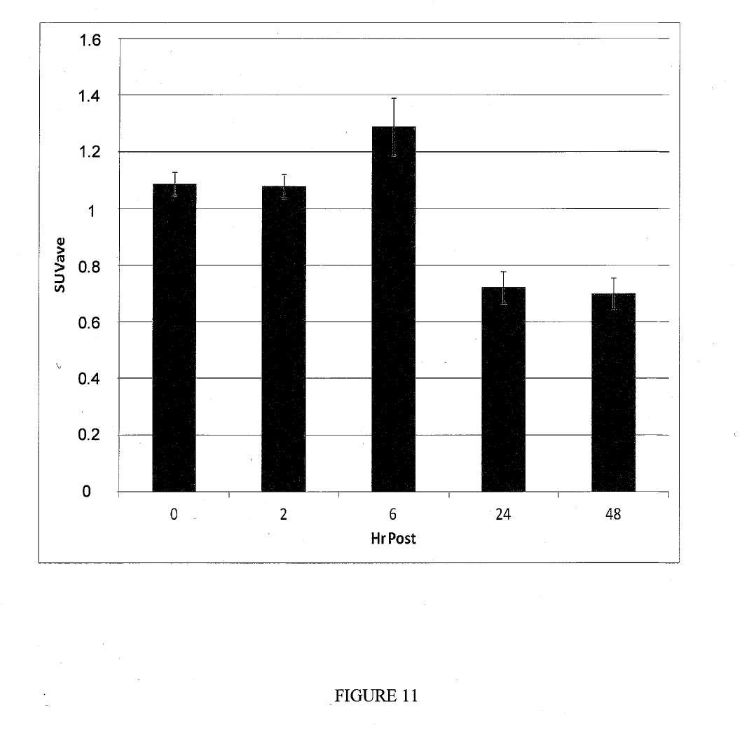

Another experiment with MLN2238 at 11 mg/kg IV was conducted in vivo with the cell line WSU-DLCL2 which is a KRAS WT cell line. This cell line showed (FIG. 11) the same trend seen with the other WT cell lines (SUVave difference between 0 and 24 or 48 h time points p-value <0.005) (8 animals per group; treated/control ˜0.44). An increase in FDG uptake was found in the earlier time points and a decrease in the uptake was found in the 24 and 48 hr groups. There was a 35% decrease in SUVave by 48 h after treatment.

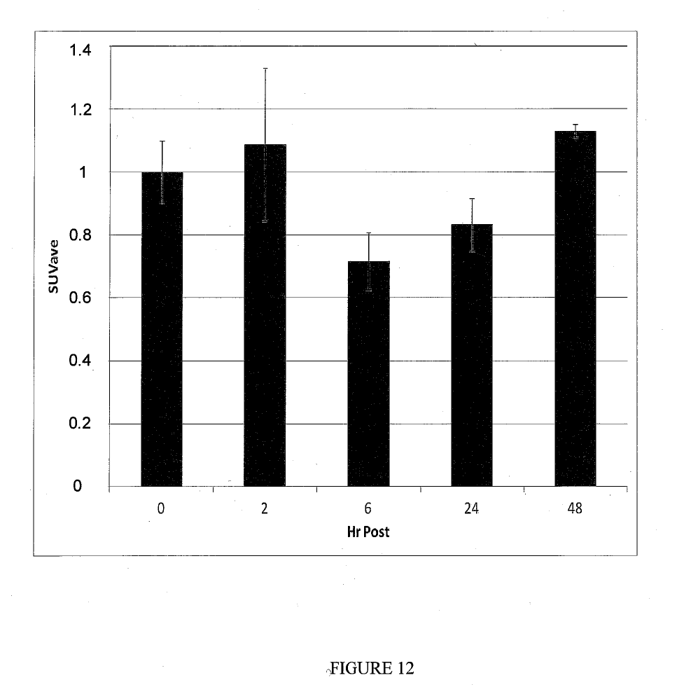

A study with the MLN2238 at 11 mg/kg IV was conducted in vivo with the primary human tumor PHTX-9C (colon tumor), G12D mutant KRAS. The FDG-PET (8 animals per group; treated/control ˜0.64) showed a decrease in the 6 h time point and an increase by 48 h (FIG. 12).

The results from some of the above studies and studies performed in a similar manner with additional cell line xenografts are compiled in the table below:

TABLE 2
Compilation of FDG-PET analyses of xenograft tumors with MLN2238 treatment

| | | | | delta of Pre vs | | | |
|---|---|---|---|---|---|---|---|
| | Basal | Endpoint | % change | post student | KRas mutant | | T/C for |
| Cell Line | SUVave | SUVave | SUVave | t-test | status | Other mutations | MLN2238 |
| PHTX-24C | 1.267 | 1.300 | 2.60 | 0.478 | mutant (A146T) | KIT(D816H) | 0.35 |
| PHTX-24C | 1.167 | 1.047 | −10.28 | 0.034 | mutant (A146T) | KIT(D816H) | 0.35 |
| PHTX-192LU | 1.427 | 1.019* | −28.59 | 0.188 | mutant (G13D) | STK11(D194N) | 0.78 |
| PHTX-192LU | 1.06 | 0.926 | −12.64 | 0.095 | mutant (G13D) | STK11(D194N) | 0.78 |
| | | | | (n = 4) | | | |
| PHTX-192LU | 0.912 | 0.653 | −28.40 | 0.010 | mutant (G13D) | STK11(D194N) | 0.78 |
| PHTX-9C | 0.832 | 0.678 | −18.51 | 0.235 | mutant (G12D) | APC(Q1367*) | 0.64 |
| H460 | 1.016 | 0.944 | −7.09 | 0.54 | mutant (Q61H) | STK11(Q37*) | 1.12 |
| | | | | | | PIK3CA(E545K) | (14 mg/kg) |
| | | | | | | MAP2K1(Y134C) | |
| PHTX-132LU | 1.109 | 0.613 | −44.72 | 0.0092 | WT | VHL(P81S) | 0.35 |
| PHTX-132LU | 0.663 | 0.546* | −17.65 | 0.451 | WT | VHL(P81S) | 0.35 |
| PHTX-132LU | 0.635 | 0.420 | −33.86 | 0.015 | WT | VHL(P81S) | 0.35 |
| WSU-DLCL2 | 1.128 | 0.700 | −37.94 | 0.0004 | WT | EZH2(Y641F) | 0.55 |
| PHTX-132LU | 0.942 | 0.453 | −51.91 | <0.0005 | WT | VHL(P81S) | 0.35 |
| H1650 | 0.846 | 0.947 | 11.94 | 0.0315 | WT | EGFR(E746_A750del) | 0.69 |
| SW48 | 1.045 | 1.218 | 16.56 | 0.039 | WT | EGFR(G2155A) | 0.46 |
| SW48 G13D | 1.112 | 1.028 | −7.55 | 0.385 | mutant (G133D) | EGFR(G2155A) | 1.03 |
| | | | | | | | (13 mg/kg) |

The endpoint measurement was at 48 h, except the values with asterisk (*), which were measured at 24 h. The studies had 8 mice in each the control and endpoint (usually 48 h) treated groups. The tumors were around 300-400 mm³ at the start of the studies. Because the controls were with each study, the student t-test was performed on the measurements. p<0.05 was judged significant. The T/C column is from separate studies with the cell line xenografts, denoting typical values for the cell lines at 21 days. Values of <0.4 are considered to represent sensitive tumors, 0.4-0.6 represent partially resistant tumors and >0.6 represent resistant tumors.

PHTX-132LU, PHTX-192LU, H1650 and H460 are lung tumors (solid tumor). WSU-DLCL2 cells are from diffuse large B-cell lymphoma (hematological tumor). The SW48, PHTX-9C and PHTX-24C are colon tumors (solid tumor). The PHTX-24C harbors a KRAS with a rare A146T mutation which may not be an activating mutation. This tumor responds to MLN2238 more like wild type KRAS tumors than the typical KRAS mutant tumors which poorly respond to MLN2238. In Table 2, tumors with wild type KRAS had significant decrease in FDG SUVave, an association found stronger in tumors with wild type KRAS and wild type EGFR. Tumors with mutant KRAS or mutant EGFR averaged typically within about 15-20% of no change (−15% to +15%) in the FDG-PET assay.

### EQUIVALENTS

Although embodiments of the invention have been described using specific terms, such description are for illustrative purposes only, and it is to be understood that changes and variations may be made without departing from the spirit or scope of the invention. Those skilled in the art will recognize, or be able to ascertain using no more than routine experimentation, many equivalents of the specific embodiments of the invention described herein. Such equivalents are intended to be encompassed by the following claims.

What is claimed:

1. A method of treating a patient having cancer, comprising the steps of
a) measuring a quantity of the cancer by a biomedical imaging technique;
b) administering a therapeutically effective amount of amount of the compound of formula (I): 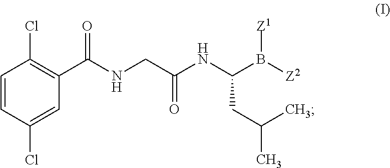
or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient;
c) repeating the biomedical imaging measurement of the quantity; and
d) proceeding with a treatment option selected from the group consisting of:
i) continuing to treat the cancer with the same dose of the compound if the quantity in c) is lower than in a);
ii) treating the cancer with a higher dose of the compound if the quantity in c) is not lower than in a); and
iii) continuing to treat the cancer with the same dose of the compound and a therapeutically effective amount of a second compound if the quantity in c) is not lower than in a).

2. The method of claim 1, wherein the biomedical imaging technique is selected from the group consisting of tomography, magnetic resonance, and ultrasound.

3. The method of claim 2, wherein the tomography is positron emission tomography (PET).

4. The method of claim 3, wherein the PET measures a quantity of the standard uptake value (SUV) and wherein the SUV is a quantity of an imaging agent selected from the group consisting of ¹⁸F-fluorodeoxyglucose (FDG), ¹⁸F-fluoro-L-thymidine (FLT), ¹¹C-acetate and ¹¹C-choline.

5. (canceled)

6. The method of claim 1, wherein the cancer comprises a solid tumor.

7. (canceled)

8. The method of claim 1, wherein the cancer comprises a hematological tumor.

9. (canceled)

10. The method of claim 1, wherein the repeat measurement is between 1 and 6 cycles of treatment with the compound.

11. The method of claim 1, wherein the repeat measurement is during the first cycle of treatment with the compound.

12.-14. (canceled)

15. The method of claim 2, wherein the magnetic resonance is magnetic resonance spectroscopy (MRS) and wherein the MRS measures a quantity of a molecule selected from the group consisting of glucose, lactate, acetate and choline.

16.-17. (canceled)

18. The method of claim 6, wherein the solid tumor has wild type KRAS or has wild type EGFR.

19. (canceled)

20. The method of claim 6, wherein the solid tumor has wild type KRAS and wild type EGFR.

21. (canceled)

22. The method of claim 1, wherein the quantity is the amount of expression of glucose transporter 4 (GLUT4).

23. (canceled)

24. The method of claim 1, wherein the compound of formula (I) is administered orally or is administered intraveneously.

25. (canceled)

26. The method of claim 1, wherein the compound of formula (I) is administered on days 1, 8, and 15 of a 28-day cycle or on days 1, 4, 8, and 11 of a 21-day cycle.

27. (canceled)

28. The method of claim 1, wherein the compound of formula (I) is characterized by formula (III-A): 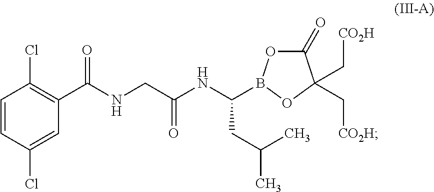 [SMILES: CC(C)C[C@H](NC(=O)CNC(=O)c1cc(Cl)ccc1Cl)B1OC(=O)C(CC(=O)O)(CC(=O)O)O1]
or a pharmaceutical composition thereof.

29. The method of claim 28, wherein the biomedical imaging technique is selected from the group consisting of tomography, magnetic resonance, and ultrasound.

30.-33. (canceled)

34. The method of claim 28, wherein the quantity is low in comparison with a quantity selected from the group consisting of a pre-determined cancer standard, a neighboring non-cancer tissue, and a pre-determined technique standard.

35. The method of claim 28, wherein the quantity is the amount of expression of glucose transporter 4 (GLUT4).

36.-37. (canceled)

38. The method of claim 28 wherein the biomedical imaging technique measures the metabolic activity of the cancer.

39.-47. (canceled)

48. The method of claim 28, wherein the cancer comprises a solid tumor.

49. (canceled)

50. The method of claim 28, wherein the cancer comprises a hematological tumor.

51. (canceled)

52. The method of claim 28, wherein the repeat measurement is between 1 and 6 cycles of treatment with the compound.

53. The method of claim 28, wherein the repeat measurement is during the first cycle of treatment with the compound.

54.-59. (canceled)

60. The method of claim 48, wherein the solid tumor has wild type KRAS or has wild type EGFR.

61. (canceled)

62. The method of claim 48, wherein the solid tumor has wild type KRAS and wild type EGFR.

63.-65. (canceled)

66. The method of claim 28, wherein the compound of formula (IIII-A) is administered orally.

67. The method of claim 66, wherein the compound of formula (III-A) is administered in one or more capsules.

68. The method of claim 28, wherein the compound of formula (III-A) is administered intraveneously.

69. The method of claim 28, wherein the compound of formula (III-A) is administered on days 1, 8, and 15 of a 28-day cycle or on days 1, 4, 8, and 11 of a 21-day cycle.

70. (canceled)

71. The method of claim 28, wherein the amount of the compound of formula (III-A) is about 2.3 mg to about 5.5 mg based on the amount of the compound of formula II.

72. A method for treating a patient having a solid tumor comprising wild type KRAS status, comprising the steps of:
a) administering to the patient a therapeutically effective amount of a proteasome inhibitor or a pharmaceutical composition thereof, wherein the solid tumor is selected from the group consisting of a lung tumor and a colon tumor;
b) monitoring the tumor activity by a biomedical imaging technique; and
c) continuing treatment with the proteasome inhibitor if the activity of the solid tumor decreases during the treatment.

73. The method of claim 72, further comprising the step of performing the biomedical imaging technique prior to administering the proteasome inhibitor.

74. The method of claim 72, wherein the biomedical imaging technique is selected from the group consisting of positron emission tomography, magnetic resonance imaging, and magnetic resonance spectroscopy.

75. (canceled)

76. The method of claim 72, wherein the activity is measured no more than 10 days after the first dose in a cycle of treatment with the proteasome inhibitor.

77. The method of claim 72, wherein the proteasome inhibitor is selected from the group consisting of a peptidyl boronic acid and a peptidyl epoxy ketone.

78. The method of claim 72, wherein the proteasome inhibitor is selected from the group consisting of bortezomib, carfilizomib, ONX-0912, and CEP-18870, or a pharmaceutically acceptable salt or pharmaceutical composition thereof.

79. (canceled)

80. The method of claim 72, wherein the solid tumor further comprises wild type EGFR status.

81. A method for treating a patient having a solid tumor comprising wild type EGFR status, comprising the step of
a) administering to the patient a therapeutically effective amount of a proteasome inhibitor or a pharmaceutical composition thereof, wherein the solid tumor is selected from the group consisting of a lung tumor and a colon tumor;
b) monitoring the tumor activity by a biomedical imaging technique; and
c) continuing treatment with the proteasome inhibitor if the activity of the solid tumor decreases during the treatment.

82. The method of claim 81, further comprising the step of performing the biomedical imaging technique prior to administering the proteasome inhibitor.

83. The method of claim 81, wherein the biomedical imaging technique is selected from the group consisting of positron emission tomography, magnetic resonance imaging, and magnetic resonance spectroscopy.

84. (canceled)

85. The method of claim 81, wherein the activity is measured no more than 10 days after the first dose in a cycle of treatment with the proteasome inhibitor.

86. The method of claim 81, wherein the proteasome inhibitor is selected from the group consisting of a peptidyl boronic acid and a peptidyl epoxy ketone.

87. The method of claim 81, wherein the proteasome inhibitor is selected from the group consisting of bortezomib, carfilizomib, ONX-0912, and CEP-18870, or a pharmaceutically acceptable salt or pharmaceutical composition thereof.

88. (canceled)

89. A method of identifying a non-hematological cancer patient who is nonresponsive to treatment with a proteasome inhibitor, comprising measuring the activity of the cancer by a biomedical imaging technique, providing a dose of the proteasome inhibitor and measuring the activity of the cancer after at least 24 hours, wherein the activity of the cancer after at least 24 hours is not changed or is changed only within about +20% to about −20% from baseline in a nonresponsive patient.

90. The method of claim 89, wherein the patient has a non-hematological cancer selected from the group consisting of lung cancer and colon cancer.

91. The method of claim 89, wherein the second measurement of activity is no more than 10 days after the dose of the proteasome inhibitor.

92. The method of claim 89, wherein the biomedical imaging technique is selected from the group consisting of positron emission tomography, magnetic resonance imaging, and magnetic resonance spectroscopy.

93. (canceled)

94. The method of claim 89, wherein the nonresponsive patient has at least one KRAS mutation in a sample comprising tumor cells from the patient.

95.-97. (canceled)

98. The method of claim 89, wherein the proteasome inhibitor is selected from the group consisting of a peptidyl boronic acid and a peptidyl epoxy ketone.

99. (canceled)

100. A method of treating a patient having cancer, comprising the steps of
a) measuring a quantity of the cancer by a biomedical imaging technique, wherein the cancer comprises a solid tumor;
b) administering a therapeutically effective amount of amount of the compound of formula (I): 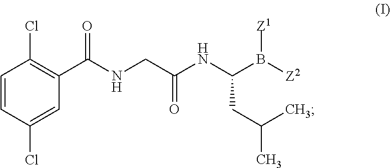
or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient;
c) repeating the biomedical imaging measurement of the quantity; and
d) proceeding with a treatment option selected from the group consisting of
i) continuing to treat the cancer with the same dose of the compound if the quantity in c) is higher than in a);
ii) treating the cancer with a higher dose of the compound if the quantity in c) is not higher than in a); and
iii) continuing to treat the cancer with the same dose of the compound and a therapeutically effective amount of a second compound if the quantity in c) is not higher than in a).

101.-102. (canceled)

103. The method of claim 100, wherein the compound of formula (I) is characterized by formula (III-A): 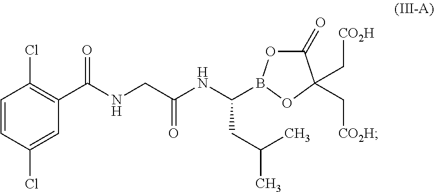 [SMILES: CC(C)C[C@H](NC(=O)CNC(=O)c1cc(Cl)ccc1Cl)B1OC(=O)C(CC(=O)O)(CC(=O)O)O1]
or a pharmaceutical composition thereof

104. The method of claim 100, wherein the repeat measurement is no more than 10 days after the dose of the compound.

105. A method of identifying a non-hematological cancer patient who is responsive to treatment with a proteasome inhibitor, comprising measuring the activity of the cancer by a biomedical imaging technique, providing a dose of the proteasome inhibitor and measuring the activity of the cancer after at least 24 hours, wherein the activity of the cancer after at least 24 hours is decreased.

106. The method of claim 105, wherein the activity is decreased more than about 20% from baseline in a responsive patient.

107. The method of claim 105, wherein the patient has a non-hematological cancer selected from the group consisting of lung cancer and colon cancer.

108. The method of claim 105, wherein the second measurement of activity is no more than 10 days after the dose of the proteasome inhibitor.

109. The method of claim 105, wherein the biomedical imaging technique is selected from the group consisting of positron emission tomography, magnetic resonance imaging, and magnetic resonance spectroscopy.

110. (canceled)

111. The method of claim 105, wherein the patient has wild type KRAS or has wild type EGFR.

112. (canceled)

113. A method of treating a patient having cancer, comprising the steps of a) measuring a quantity of a feature of the cancer by a biomedical imaging technique, wherein the cancer comprises a solid tumor, wherein the feature is selected from the group consisting of tumor surface appearance, metabolic activity and metabolic capacity; and
b) administering a therapeutically effective amount of a compound of formula (I): 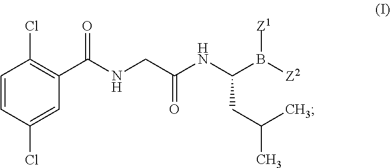
or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to the patient if the quantity is low.

114. The method of claim 113, wherein the biomedical imaging technique is selected from the group consisting of tomography, magnetic resonance, and ultrasound.

115. The method of claim 113, wherein the solid tumor has wild type KRAS or has wild type EGFR.

116. (canceled)

117. The method of claim 113, wherein the solid tumor has wild type KRAS and wild type EGFR.

118.-119. (canceled)

120. The method of claim 113, wherein the quantity is low in comparison with a quantity selected from the group consisting of a pre-determined cancer standard, a neighboring non-cancer tissue, and a pre-determined technique standard.

121. The method of claim 120, wherein the feature is metabolic capacity and the quantity is glucose transporter 4 (GLUT4) expression.

122.-130. (canceled)
